(19) United States
(12) Patent Application Publication
(10) Pub. No.: US 2023/0141382 A1
(43) Pub. Date: May 11, 2023

(54) COMPOSITIONS FOR PRESERVATION OF VIRAL VECTORS

(71) Applicant: CELLULAR BIOMEDICINE GROUP INC., Rockville, MD (US)

(72) Inventors: Shuqian YU, Hong Kong (CN); Junfeng WU, Hong Kong (CN); Yuhong XIA, Hong Kong (CN); Luyi ZHANG, Hong Kong (CN); Li ZHANG, Hong Kong (CN); Ting YAN, Hong Kong (CN); Ting HAN, Hong Kong (CN)

(21) Appl. No.: 17/918,764

(22) PCT Filed: Apr. 23, 2021
(86) PCT No.: PCT/US2021/028824

§ 371 Date: Oct. 13, 2022

(30) Foreign Application Priority Data
Apr. 23, 2020 (CN) 2020103287013

(51) Int. Cl. C12N 15/86; C12N 1/04

(52) U.S. Cl. CPC C12N 15/86; C12N 1/04; C12N 2740/16043

(57) ABSTRACT

Compositions and methods for the preservation and long-term storage of viral vectors such as lentiviral vectors are provided.

### TECHNICAL FIELD

The present disclosure relates generally to the field of stabilizing and preserving biological materials. In particular, the present disclosure relates to compositions and methods for the preservation and long-term storage of viral vectors such as lentiviral vectors.

### BACKGROUND OF THE DISCLOSURE

Viral vectors are currently one of the most commonly used methods of gene introduction. By means of modifying a viral genome, a viral vector can carry a heterologous sequence and related viral elements, which are then packaged into a viral particle. Subsequently, the virus is used to transfect a host, thereby expressing the heterologous sequence in the host.

Lentiviruses (LV) are RNA viruses and belong to the Retrovidae family. An important feature of this type of viruses is that the virion contains an RNA-dependent polymerase or reverse transcriptase. The primary cells infected by lentiviruses are mainly lymphocytes and macrophages. Lentiviruses, such as human immunodeficiency virus (HIV), simian immunodeficiency virus (SIV), etc., have a long incubation period before infection and diseases.

Among lentiviral vectors, the vector system that is developed based on the human immunodeficiency type 1 virus (HIV-1) has the most in-depth studies and is most widely applied. It can efficiently introduce a target gene into animal cells. The lentiviral genome is composed of positive-strand RNA. After the genome enters the cell, it is reversed into DNA in the cytoplasm by its own reverse transcriptase to form a pre-DNA integration complex, which can enter the nucleus to integrate the DNA into the cell genome for transcription and expression.

Compared with other commonly used viral vectors (such as adenoviral vectors that cannot be integrated into chromosomes), lentiviral vectors have significant advantages such as being able to infect non-dividing cells, carrying large foreign gene fragments, and being able to express for a long time.

In the process of preparing chimeric antigen receptor T cells (CAR-T cells), a chimeric antigen receptor gene can be introduced into a patient's autologous T cells by means of lentiviral transduction; in addition, after in vitro expansion, a chimeric antigen receptor T cell injection for clinical use can be obtained.

Transduction potential and potency of lentivirus is influenced by various factors, including temperature, pH, freeze-and-thaw frequency and incubation conditions in growth media and serum components during vector production and storage. Lentivirus titer may decrease with increased freeze/thaw cycles and storage at higher temperatures (Kigashikawa and Chang 2001, Virology 280, 124-131). In order for therapies to be most effective, it is desirable to have lentiviral vectors that maintain their potency. Therefore, the long-term storage stability of lentiviral vectors can affect the efficacy of clinical immunotherapy.

### SUMMARY

The present disclosure provides for a preservation composition (also referred to as “combination reagent” herein) for preserving viral vectors. The preservation composition may comprise a first preservation reagent (also referred to as “component A” herein) and a second preservation reagent (also referred to as “component B” herein).

The first preservation reagent may comprise (or consist of, or consist essentially of) sucrose at a concentration of 100 g/L to 250 g/L, potassium dihydrogen phosphate or sodium dihydrogen phosphate at a concentration of 0.8 mmol/L (mM) to 1.2 mmol/L (mM), disodium hydrogen phosphate or dipotassium hydrogen phosphate at a concentration of 2.6 mmol/L-3.2 mmol/L, and sodium chloride at a concentration of 8.5-10 g/L.

The second preservation reagent may comprise (or consist of, or consist essentially of) sucrose at a concentration of 100 g/L to 250 g/L, albumin (e.g., human albumin) at a concentration of 40 g/L to 300 g/L, sodium caprylate or potassium caprylate at a concentration of 4 mmol/L to 20 mmol/L, sodium N-acetyltryptophan or potassium N-acetyltryptophan at a concentration of 4 mmol/L to 20 mmol/L, potassium dihydrogen phosphate or sodium dihydrogen phosphate at a concentration of 0.1 mM-1.2 mM, disodium hydrogen phosphate or dipotassium hydrogen phosphate at a concentration of 0.5 mM-3 mM, and sodium chloride at a concentration of 1.5-10 g/L.

The preservation composition may comprise (or consist of, or consist essentially of) sucrose at a concentration of 100 g/L to 250 g/L, albumin at a concentration of 20 g/L to 150 g/L, sodium caprylate or potassium caprylate at a concentration of 1.6 mmol/L to 10 mmol/L, sodium N-acetyltryptophan or potassium N-acetyltryptophan at a concentration of 1.6 mmol/L to 10 mmol/L, potassium dihydrogen phosphate or sodium dihydrogen phosphate at a concentration of 0.5 mM to 1 mM, disodium hydrogen phosphate or dipotassium hydrogen phosphate at a concentration of 1.6 mM to 3 mM, and sodium chloride at a concentration of 5.5-10 g/L.

The first preservation reagent and the second preservation reagent may be combined at a volume ratio ranging from about 1:1 to about 4:1.

In the preservation composition, the first preservation reagent and the second preservation reagent may be separate. The preservation composition may comprise (or consist of, or consist essentially of) a mixture of the first preservation reagent and the second preservation reagent.

The preservation composition may have an osmotic pressure ranging from about 900 mOsmol/kg to about 1400 mOsmol/kg.

The preservation composition (or the first preservation reagent, or the second preservation reagent) may have a pH ranging from about 6.5 to about 7.5, from about 6.8 to about 7.5, from about 7 to about 7.4, from about 6.95 to about 7.45.

The preservation composition may further comprise viral vectors, such as retroviral vectors (e.g., lentiviral vectors). The lentiviral vectors may be human immunodeficiency virus (HIV); visna-maedi virus (VMV); caprine arthritis-encephalitis virus (CAEV); equine infectious anemia virus (EIAV); feline immunodeficiency virus (FIV); bovine immunodeficiency virus (BIV); or simian immunodeficiency virus (SIV). In certain embodiments, the lentiviral vectors are HIV-1 or HIV-2.

The present disclosure provides for a method for preserving viral vectors. The method may comprise combining the preservation composition with the viral vectors.

Also encompassed by the present disclosure is a method for preserving viral vectors using the preservation composition. The method may comprise: (a) combining the first preservation reagent with the viral vectors to form a mixture (or combination); and (b) combining the second preservation reagent with the mixture (or combination) of step (a). The first preservation reagent and the second preservation reagent may be combined at a volume ratio ranging from about 1:1 to about 4:1.

The present disclosure provides for a method of transducing a cell. The method may comprise contacting the cell with the preservation composition which contains the viral vectors.

The cell may be a mammalian cell, such as a human cell. The cell may be a stem cell or progenitor cell. The cell may be a hematopoietic cell, such as a lymphocyte, including a T lymphocyte and a B lymphocyte.

One object of the present disclosure is to provide a clinical-grade, low-endotoxic viral vector preservation combination reagent for long-term storage of lentiviral vectors.

In a first aspect, the present disclosure provides a combination reagent, and the combination reagent includes component A and component B, where component A includes: sucrose, potassium dihydrogen phosphate (KH₂PO₄), disodium hydrogen phosphate heptahydrate (Na₂HPO₄.7H₂O), sodium chloride and water; component B includes: sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), sodium chloride and water.

In one embodiment, in component A, a concentration of sucrose is 10-25 g/100 mL, a concentration of potassium dihydrogen phosphate (KH₂PO₄) is 120-150 mg/L, a concentration of disodium hydrogen phosphate heptahydrate (Na₂HPO₄.7H₂O) is 700-850 mg/L, and a concentration of sodium chloride is 8500-10000 mg/L.

In one embodiment, component A has a pH of 6.95-7.45, 7-7.45 or 7.05-7.35.

In one embodiment, in component B, a concentration of sucrose is 10-25 g/100 mL, a concentration of human albumin is 4-30 g/100 mL, a concentration of sodium caprylate is 4-20 mmol/L, a concentration of sodium N-acetyltryptophan is 4-20 mmol/L, a concentration of potassium dihydrogen phosphate (KH₂PO₄) is 20-150 mg/L, a concentration of disodium hydrogen phosphate heptahydrate (Na₂HPO₄.7H₂O) is 150-800 mg/L, and a concentration of sodium chloride is 1500-10000 mg/L.

In one embodiment, component B has a pH of 6.95-7.45, 7-7.45 or 7.05-7.35.

In one embodiment, a volume ratio of component A:component B, when in use, is within a range of from 4:1 to 1:1.

In a second aspect, the present disclosure provides the use of the combination reagent provided in the first aspect in the preservation of lentiviral vectors.

In a third aspect, the present disclosure provides the use of the combination reagent provided in the first aspect of a reagent for preserving lentiviral vectors.

In a fourth aspect, the present disclosure provides a method for preserving lentiviral vectors; the method uses the combination reagent provided in the first aspect to preserve lentiviral vectors.

In one embodiment, the method includes the following steps: (i) adding component A during a production and purification process of a lentiviral vector; (ii) adding component B prior to aliquoting to finally form a preservation solution of the lentiviral vector.

In one embodiment, a volume ratio of component A:component B is within a range of from 4:1 to 1:1.

In one embodiment, in component A, a concentration of sucrose is 10-25 g/100 mL, a concentration of potassium dihydrogen phosphate (KH₂PO₄) is 120-150 mg/L, a concentration of disodium hydrogen phosphate heptahydrate (Na₂HPO₄.7H₂O) is 700-850 mg/L, and a concentration of sodium chloride is 8500-10000 mg/L.

In one embodiment, in component B, a concentration of sucrose is 10-25 g/100 mL, a concentration of human albumin is 4-30 g/100 mL, a concentration of sodium caprylate is 4-20 mmol/L, a concentration of sodium N-acetyltryptophan is 4-20 mmol/L, a concentration of potassium dihydrogen phosphate (KH₂PO₄) is 20-150 mg/L, a concentration of disodium hydrogen phosphate heptahydrate (Na₂HPO₄.7H₂O) is 150-800 mg/L, and a concentration of sodium chloride is 1500-10000 mg/L.

In one embodiment, component A has a pH of 6.95-7.45, 7-7.45 or 7.05-7.35.

In one embodiment, component B has a pH of 6.95-7.45, 7-7.45 or 7.05-7.35.

In one embodiment, the preservation solution has an osmotic pressure of 900-1400 mOsmol/Kg.

In one embodiment, the preservation solution has a pH of 6.95-7.45, 7-7.45 or 7.05-7.35.

In one embodiment, in the preservation solution, the concentration of sucrose is 10-25 g/100 mL, the concentration of human albumin is 2-15 g/100 mL, the concentration of sodium caprylate is 1.6-10 mmol/L, the concentration of sodium N-acetyltryptophan is 1.6-10 mmol/L, the concentration of potassium dihydrogen phosphate (KH₂PO₄) is 70-130 mg/L, the concentration of disodium hydrogen phosphate heptahydrate (Na₂HPO₄.7H₂O) is 450-800 mg/L, and the concentration of sodium chloride is 5500-10000 mg/L.

In a fifth aspect, the present disclosure provides a preservation solution for lentiviral vectors. The preservation solution includes sucrose, potassium dihydrogen phosphate (KH₂PO₄), disodium hydrogen phosphate heptahydrate (Na₂HPO₄.7H₂O), sodium chloride, human albumin, sodium caprylate, sodium N-acetyl tryptophan, and water, where the concentration of sucrose is 10-25 g/100 mL, the concentration of human albumin is 2-15 g/100 mL, the concentration of sodium caprylate is 1.6-10 mmol/L, the concentration of sodium N-acetyltryptophan is 1.6-10 mmol/L, the concentration of potassium dihydrogen phosphate (KH₂PO₄) is 70-130 mg/L, the concentration of disodium hydrogen phosphate heptahydrate (Na₂HPO₄.7H₂O) is 450-800 mg/L, and the concentration of sodium chloride is 5500-10000 mg/L.

In one embodiment, the preservation solution has an osmotic pressure of 900-1400 mOsmol/Kg.

In one embodiment, the preservation solution has a pH of 6.95-7.45, 7-7.45 or 7.05-7.35.

The clinical-grade, low-endotoxic combination reagent for viral vector preservation provided by the present disclosure is suitable for the production process of GMP-grade lentiviral vectors, can prolong the validity period of lentiviral preservation, and leads to a stable preparation process of high-purity and high-quality recombinant lentiviral vectors. It can be used to perform linear amplification, stably and continuously improve the production process, effectively control the cost, and perfect the production technology. In addition, it can help the preparation and clinical use of CAR-T cell products.

### DETAILED DESCRIPTION

The present disclosure provides compositions and methods for stabilizing/preservation of viral vectors. The preservation compositions help maintain infectious titer and/or expression of heterologous sequences when stored for various periods of time across a broad range of temperatures, and/or throughout multiple freeze thaw cycles.

Viral vectors such as lentiviruses may be affected negatively by storage conditions, such as storage at 4° C. for long periods of time or experiencing multiple freeze/thaw cycles after storage at lower temperatures such as −80° C. Lentiviral titers have been observed to decrease with increasing freeze/thaw cycles and increasing temperature. The present preservation compositions maintain the potency of viral vectors under various temperatures and time periods compared to viral vectors not stored in these preservation compositions.

The present disclosure provides for a preservation composition that promotes survival and retention of viability of viral vectors for storage. The present compositions and methods may be used for hypothermic preservation (e.g., between about 4° C. and about 10° C.) or for cryopreservation, including freezing and lyophilization.

The biological material can then be used in a variety of research and clinical settings, for example, for immunotherapies, gene therapies, cell-based therapeutics, or vaccination.

The present disclosure provides for a preservation composition (also referred to as “combination reagent” herein) for preserving viral vectors. The preservation composition may comprise a first preservation reagent (also referred to as “component A” herein) and a second preservation reagent (also referred to as “component B” herein).

The first preservation reagent may comprise (or consist of, or consist essentially of) one or more saccharides (e.g., sucrose) at a concentration of 100 g/L to 250 g/L, potassium dihydrogen phosphate or sodium dihydrogen phosphate at a concentration of 0.8 mmol/L (mM) to 1.2 mmol/L (mM), disodium hydrogen phosphate or dipotassium hydrogen phosphate at a concentration of 2.6 mmol/L-3.2 mmol/L, and sodium chloride at a concentration of 8.5-10 g/L.

The second preservation reagent may comprise (or consist of, or consist essentially of) one or more saccharides (e.g., sucrose) at a concentration of 100 g/L to 250 g/L, one or more macromolecules (e.g., albumin such as human albumin) at a concentration of 40 g/L to 300 g/L, sodium caprylate or potassium caprylate at a concentration of 4 mmol/L to 20 mmol/L, sodium N-acetyltryptophan or potassium N-acetyltryptophan at a concentration of 4 mmol/L to 20 mmol/L, potassium dihydrogen phosphate or sodium dihydrogen phosphate at a concentration of 0.1 mM-1.2 mM, disodium hydrogen phosphate or dipotassium hydrogen phosphate at a concentration of is 0.5 mM-3 mM, and sodium chloride at a concentration of 1.5-10 g/L.

The preservation composition may comprise (or consist of, or consist essentially of) one or more saccharides (e.g., sucrose) at a concentration of 100 g/L to 250 g/L, one or more macromolecules (e.g., albumin such as human albumin) at a concentration of 20 g/L to 150 g/L, sodium caprylate or potassium caprylate at a concentration of 1.6 mmol/L to 10 mmol/L, sodium N-acetyltryptophan or potassium N-acetyltryptophan at a concentration of 1.6 mmol/L to 10 mmol/L, potassium dihydrogen phosphate or sodium dihydrogen phosphate at a concentration of 0.5 mM to 1 mM, disodium hydrogen phosphate or dipotassium hydrogen phosphate at a concentration of 1.6 mM to 3 mM, and sodium chloride at a concentration of 5.5-10 g/L.

The first preservation reagent and the second preservation reagent may be combined at a volume ratio ranging from about 1:1 to about 4:1.

In the preservation composition, the first preservation reagent and the second preservation reagent may be separate. The preservation composition may comprise (or consist of, or consist essentially of) a mixture of the first preservation reagent and the second preservation reagent.

In certain embodiments, the present composition further comprises viral vectors or viral particles. In certain embodiments, the present composition does not comprise viral vectors or viral particles.

The present disclosure provides for a method for preserving viral vectors or viral particles.

The method may comprise the steps of: (a) contacting/mixing/combining the viral vectors or viral particles with a first preservation reagent to form a first mixture or combination, (b) contacting/mixing/combining the first mixture/combination with a second preservation reagent to form a second mixture or combination, and (c) optionally storing or freezing the second mixture or combination for storage.

The method may comprise the steps of: (a) contacting/mixing/combining the viral vectors or viral particles with a preservation composition (e.g., to form a mixture or to form a combination), and (b) optionally storing or freezing the mixture or the combination of the preservation composition and the viral vectors or viral particles.

The viral vectors may be stored/preserved at any suitable temperature, e.g., ranging from about −80° C. to about 25° C., from about −70° C. and −200° C., from about −20° C. to about 18° C., from about −20° C. to about 4° C., from about 4° C. to about 25° C., from about 4° C. to about 18° C., at or below about 37° C., at or below about 25° C., at or below about 20° C., at or below about 10° C., at or below about 8° C., at or below about 4° C., at or below about 0° C., at or below about −20° C., at or below about −60° C., at or below about −70° C., at or below about −80° C., at or below about −135° C., at or below about −196° C., at or below about −100° C., at or below about −196° C. (e.g., liquid nitrogen), at or below about −205° C. (e.g., slush nitrogen which is a mixture of liquid and solid nitrogen), or in liquid nitrogen, for at least or about 3 hours, at least or about 6 hours, at least or about 12 hours, at least or about 24 hours, at least or about 48 hours, at least or about 72 hours, at least or about 96 hours, at least or about 1 week, at least or about 2 weeks, at least or about 3 weeks, at least or about 1 month, at least or about 2 months, at least or about 3 months, at least or about 4 months, at least or about 5 months, at least about or 6 months, at least or about 1 year, at least or about 2 years or more or any time period.

The present compositions stabilize viral vectors at a wide range of temperatures for extended periods of time. In certain embodiments, the composition stabilizes the viral vector at about 20° C., at about 10° C., at about 4° C., at about 0° C., at about −5° C., at about −10° C., at about −20° C., at about −30° C., at about −40° C., at about −50° C., at about −60° C., at about −70° C., at about −80° C., or any temperature in between (and described herein) for any time period including an extended time period. In certain embodiments, the viral vector is stable for at least or about 24 hours, at least or about 48 hours, at least or about 72 hours, at least or about 96 hours, at least or about 1 week, at least or about 2 weeks, at least or about 3 weeks, at least or about 1 month, at least or about 2 months, at least or about 3 months, at least or about 4 months, at least or about 5 months, at least about or 6 months, at least or about 1 year, at least or about 2 years or more or any time period. One of skill in the art will understand that temperatures above and below the listed temperatures, and time periods shorter and longer than the listed time periods, may apply.

The present compositions stabilize viral vectors after freezing and thawing the vectors. In one embodiment, the composition stabilizes viral vectors for 1, 2, 3, 4, 5, 6, 7, 8, 9, 10 or more freeze/thaw cycles at various temperatures disclosed herein, e.g., −20° C., −60° C., −70° C., −80° C., etc. In one embodiment, when preserved/stabilized using the present preservation compositions, the titer of the viral vectors is stable for one or more freeze/thaw cycles. In another embodiment, the titer of the viral vectors is stable for two or more freeze/thaw cycles. In another embodiment, the titer of the viral vectors is stable for three or more freeze/thaw cycles.

The present method may further comprise the step of (c) thawing the frozen mixture or the combination of the preservation composition and the viral vectors or viral particles.

In certain embodiments, after preservation using the present preservation compositions, the viral vectors have a titer that is at least or about 50%, at least or about 55%, at least or about 60%, at least or about 65%, at least or about 70%, at least or about 75%, at least or about 80%, at least or about 85%, at least or about 90%, or at least or about 95%, of the viral titer before preservation.

In certain embodiments, the viral vector has a titer that is stable for more than one month, for more than six months, for more than one year, when the composition is stored/preserved at the temperature discussed herein.

To “preserve” refers to the use of preservation compositions to essentially maintain the biological activity or integrity of a biological material. For example, the infectious titer of and/or expression of heterologous sequences in a viral vector can be preserved with the present preservation compositions. In particular embodiments, a viral vector is preserved at a temperature when its titer is maintained for a duration of time at that temperature or where the titer decreases less than or about 5%, less than or about 10%, less than or about 15%, less than or about 20%, less than or about 25%, less than or about 30%, less than or about 35%, less than or about 40%, less than or about 45%, less than or about 50%, less than or about 55%, less than or about 60%, less than or about 65%, less than or about 70%, less than or about 75%, less than or about 80%, less than or about 85%, less than or about 90%, or any intervening value, compared to the viral titer before preservation.

A “stable” composition is one in which the viral vector therein substantially retains its infectious titer, vector particle to infectivity ratio, and/or expression of heterologous sequences upon storage at a temperature for a period of time.

In particular embodiments, the viral vector is stable at a temperature when its infectious titer is maintained for a duration of time (e.g., hours, days, weeks, months, years) at that temperature or where the titer after preservation is greater than or about 5%, greater than or about 10%, greater than or about 15%, greater than or about 20%, greater than or about 25%, greater than or about 30%, greater than or about 35%, greater than or about 40%, greater than or about 45%, greater than or about 50%, greater than or about 55%, greater than or about 60%, greater than or about 65%, greater than or about 65%, greater than or about 70%, greater than or about 75%, greater than or about 80%, greater than or about 85%, greater than or about 90%, or any intervening value, of the viral titer before preservation.

In certain embodiments, the viral vector is stable at a temperature when the vector particle to infectivity (P:I) ratio of the virus is about 5%, about 10%, within about 15%, about 20%, about 25%, about 30%, about 35%, about 40%, about 45%, about 50%, about 55%, about 60%, about 65%, about 70%, about 75%, about 80%, about 85%, about 90%, about 95%, or any intervening value of the original or first measured (P:I) of the virus before preservation. In one embodiment, the viral vector is stable at a temperature when the vector particle to infectivity (P:I) ratio of the virus is within about 50% or more of the original or first measured (P:I) of the virus before preservation.

The present disclosure provides for a method for preserving (e.g., cryopreserving) viral vectors or viral particles. The method may comprise the following steps: (a) combining/mixing/contacting the present preservation composition with viral vectors or viral particles; (b) cooling and/or freezing the mixture; and (c) storing the mixture (e.g., at appropriate storing conditions).

The viral vectors or viral particles may be added to the preservation composition (or the first preservation reagent). Alternatively, the preservation composition (or the first preservation reagent) can be added to the viral vectors or viral particles. In certain embodiments, in step (a) of the method, the viral vectors or viral particles are suspended in the preservation composition.

In certain embodiments, the preservation composition is a hypothermic preservation composition (e.g., a hypothermic preservation solution). In certain embodiments, the preservation composition is a cryopreservation composition (e.g., a cryopreservation solution). In such case the solution may be for example a freezing solution (in which case the viral vectors or viral particles are frozen) or a lyophilization solution (in which case the viral vectors or viral particles are lyophilized).

In certain embodiments, the present cryopreservation composition (with or without viral vectors or viral particles) is in a cryopreserved state (or a frozen state), or has been thawed from a cryopreserved state. In certain embodiments, the present cryopreservation composition (with or without viral vectors or viral particles) is in a hypothermic state, or in a freeze-dried state.

The present preservation composition may be a liquid or a solid. In certain embodiments, the present preservation composition is a concentrate composition, such as, in a dry form (e.g., powder, tablet, granular or any other suitable physical form) or in liquid form as, e.g., 2×, 3×, 4×, 5×, 6×, 7×, 8×, 9×, 10×, 15×, 20× etc. stock solutions. The stock solutions can be diluted 2×, 3×, 4×, 5×, 6×, 7×, 8×, 9×, 10×, 15×, 20× etc. by, e.g., a culture medium, a physiologic solution, a buffer, water etc. The dry form of the preservation composition may be converted to a liquid form by adding, e.g., a culture medium, a physiologic solution, a buffer, water etc. (e.g., dissolved in, e.g., a culture medium, a physiologic solution, a buffer, water etc.).

In certain embodiment, the concentrations of the components discussed herein are the concentrations of the components in a stock solution of the present preservation composition. In certain embodiment, the concentrations of the components discussed herein are the concentrations of the components in a working solution of the present preservation composition.

The present preservation composition may be a solution. In certain embodiments, the composition is an aqueous solution of the components discussed herein.

In certain embodiments, the present preservation composition (or the first preservation reagent, or the second preservation reagent) has a pH ranging from about 6.0 to about 8.5, from about 6.5 to about 8, from about 6.6 to about 7.5, from about 6.8 to about 7.6, from about 6.9 to about 7.5, from about 6.95 to about 7.45, from about 7 to about 7.45, from about 7.05 to about 7.35, from about 7.35 to about 7.45, or from about 7.2 to about 7.4, at room temperature or ambient temperature (for example, at 25° C.).

The present composition can be obtained in a solid form by mixing the components discussed herein, or as an aqueous solution by dissolving the components in water, a buffer, a solution, a culture medium, etc.

Saccharides include oligosaccharides such as monosaccharides and disaccharides, polysaccharides, and the like. Saccharides include sugars. Non-limiting examples of saccharides include sucrose, sorbitol, glucose, fructose, galactose, trehalose, mannose, raffinose, stachyose, dextran, xylose, arabinose, mannitol, xylitol, myo-inositol, lactose, maltose, cellobiose, lactitol, maltitol, methyl cellulose, carboxymethyl cellulose, glycogen, amylose, amylopectin, inulin, sodium alginate, ethyl cellulose, hydroxyethyl cellulose, xanthan gum, glucosamine, galactosamine, and combinations thereof. In certain embodiments, the saccharide is sucrose.

In certain embodiments, the macromolecule comprises albumin, gelatin, or a combination thereof. Non-limiting examples of albumin include serum albumin (e.g., human serum albumin or HAS), plasma albumin (e.g., human plasma albumin), bovine serum albumin, and/or synthetic serum albumin), ovalbumin, plant albumin, or combinations thereof. In one embodiment, the albumin is human albumin.

In certain embodiments, for the preservation composition, the volume ratio of the first preservation reagent to the second preservation reagent may range from about 8:1 to about 1:3, from about 6:1 to about 1:3, from about 5:1 to about 1:3, from about 4:1 to about 1:3, from about 3:1 to about 1:3, from about 2:1 to about 1:3, from about 8:1 to about 1:2, from about 6:1 to about 1:2, from about 5:1 to about 1:2, from about 4:1 to about 1:2, from about 3:1 to about 1:2, from about 2:1 to about 1:2, from about 8:1 to about 1:1, from about 6:1 to about 1:1, from about 5:1 to about 1:1, from about 4:1 to about 1:1, from about 3:1 to about 1:1, from about 2:1 to about 1:1, about 4:1, about 3:1, about 2:1, or about 1:1.

The preservation composition may have any suitable osmotic pressure or osmolarity, for example, from about 400 mOsmol/kg to about 2500 mOsmol/kg, from about 400 mOsmol/kg to about 2000 mOsmol/kg, from about 400 mOsmol/kg to about 1800 mOsmol/kg, from about 400 mOsmol/kg to about 1600 mOsmol/kg, from about 400 mOsmol/kg to about 1500 mOsmol/kg, from about 400 mOsmol/kg to about 1400 mOsmol/kg, from about 400 mOsmol/kg to about 1200 mOsmol/kg, from about 400 mOsmol/kg to about 1000 mOsmol/kg, from about 500 mOsmol/kg to about 2500 mOsmol/kg, from about 500 mOsmol/kg to about 2000 mOsmol/kg, from about 500 mOsmol/kg to about 1800 mOsmol/kg, from about 500 mOsmol/kg to about 1600 mOsmol/kg, from about 500 mOsmol/kg to about 1500 mOsmol/kg, from about 500 mOsmol/kg to about 1400 mOsmol/kg, from about 500 mOsmol/kg to about 1200 mOsmol/kg, from about 500 mOsmol/kg to about 1000 mOsmol/kg, from about 600 mOsmol/kg to about 2500 mOsmol/kg, from about 600 mOsmol/kg to about 2000 mOsmol/kg, from about 600 mOsmol/kg to about 1800 mOsmol/kg, from about 600 mOsmol/kg to about 1600 mOsmol/kg, from about 600 mOsmol/kg to about 1500 mOsmol/kg, from about 600 mOsmol/kg to about 1400 mOsmol/kg, from about 600 mOsmol/kg to about 1200 mOsmol/kg, from about 600 mOsmol/kg to about 1000 mOsmol/kg, from about 700 mOsmol/kg to about 2500 mOsmol/kg, from about 700 mOsmol/kg to about 2000 mOsmol/kg, from about 700 mOsmol/kg to about 1800 mOsmol/kg, from about 700 mOsmol/kg to about 1600 mOsmol/kg, from about 700 mOsmol/kg to about 1500 mOsmol/kg, from about 700 mOsmol/kg to about 1400 mOsmol/kg, from about 700 mOsmol/kg to about 1200 mOsmol/kg, from about 700 mOsmol/kg to about 1000 mOsmol/kg, from about 800 mOsmol/kg to about 2500 mOsmol/kg, from about 800 mOsmol/kg to about 2000 mOsmol/kg, from about 800 mOsmol/kg to about 1800 mOsmol/kg, from about 800 mOsmol/kg to about 1600 mOsmol/kg, from about 800 mOsmol/kg to about 1500 mOsmol/kg, from about 800 mOsmol/kg to about 1400 mOsmol/kg, from about 800 mOsmol/kg to about 1200 mOsmol/kg, from about 800 mOsmol/kg to about 1000 mOsmol/kg, from about 900 mOsmol/kg to about 2500 mOsmol/kg, from about 900 mOsmol/kg to about 2000 mOsmol/kg, from about 900 mOsmol/kg to about 1800 mOsmol/kg, from about 900 mOsmol/kg to about 1600 mOsmol/kg, from about 900 mOsmol/kg to about 1500 mOsmol/kg, from about 900 mOsmol/kg to about 1400 mOsmol/kg, from about 900 mOsmol/kg to about 1200 mOsmol/kg, from about 900 mOsmol/kg to about 1000 mOsmol/kg, greater than about 500 mOsmol/kg, greater than about 600 mOsmol/kg, greater than about 700 mOsmol/kg, greater than about 800 mOsmol/kg, less than about 2000 mOsmol/kg, less than about 1500 mOsmol/kg, less than about 1400 mOsmol/kg, or less than about 1600 mOsmol/kg.

In certain embodiments, the preservation composition (or the first preservation reagent, or the second preservation reagent) comprises one or more saccharides (e.g., sucrose) at a concentration ranging from about 1 g/100 mL to about 50 g/100 mL, from about 5 g/100 mL to about 50 g/100 mL, from about 5 g/100 mL to about 40 g/100 mL, from about 5 g/100 mL to about 35 g/100 mL, from about 5 g/100 mL to about 30 g/100 mL, from about 5 g/100 mL to about 25 g/100 mL, from about 5 g/100 mL to about 20 g/100 mL, from about 5 g/100 mL to about 15 g/100 mL, from about 5 g/100 mL to about 10 g/100 mL, from about 15 g/100 mL to about 50 g/100 mL, from about 15 g/100 mL to about 40 g/100 mL, from about 15 g/100 mL to about 35 g/100 mL, from about 15 g/100 mL to about 30 g/100 mL, from about 15 g/100 mL to about 25 g/100 mL, from about 15 g/100 mL to about 20 g/100 mL, from about 20 g/100 mL to about 50 g/100 mL, from about 20 g/100 mL to about 40 g/100 mL, from about 20 g/100 mL to about 35 g/100 mL, from about 20 g/100 mL to about 30 g/100 mL, from about 20 g/100 mL to about 25 g/100 mL, from about 10 g/100 mL to about 50 g/100 mL, from about 10 g/100 mL to about 45 g/100 mL, from about 10 g/100 mL to about 40 g/100 mL, from about 10 g/100 mL to about 35 g/100 mL, from about 10 g/100 mL to about 30 g/100 mL, from about 10 g/100 mL to about 20 g/100 mL, from about 10 g/100 mL to about 15 g/100 mL, from about 10 g/100 mL to about 25 g/100 mL, about 40 g/100 mL, about 35 g/100 mL, about 30 g/100 mL, about 25 g/100 mL, about 5 g/100 mL, about 1 g/100 mL, about 20 g/100 mL, about 15 g/100 mL, or about 10 g/100 mL.

In certain embodiments, the composition (or the first preservation reagent, or the second preservation reagent) comprises one or more saccharides (e.g., sucrose) at a concentration ranging from about 10 g/L to about 500 g/L, from about 50 g/L to about 500 g/L, from about 50 g/L to about 400 g/L, from about 50 g/L to about 350 g/L, from about 50 g/L to about 300 g/L, from about 50 g/L to about 250 g/L, from about 50 g/L to about 200 g/L, from about 50 g/L to about 150 g/L, from about 50 g/L to about 100 g/L, from about 150 g/L to about 500 g/L, from about 150 g/L to about 400 g/L, from about 150 g/L to about 350 g/L, from about 150 g/L to about 300 g/L, from about 150 g/L to about 250 g/L, from about 150 g/L to about 200 g/L, from about 200 g/L to about 500 g/L, from about 200 g/L to about 400 g/L, from about 200 g/L to about 350 g/L, from about 200 g/L to about 300 g/L, from about 200 g/L to about 250 g/L, from about 100 g/L to about 500 g/L, from about 100 g/L to about 450 g/L, from about 100 g/L to about 400 g/L, from about 100 g/L to about 350 g/L, from about 100 g/L to about 300 g/L, from about 100 g/L to about 200 g/L, from about 100 g/L to about 150 g/L, from about 100 g/L to about 250 g/L, about 400 g/L, about 350 g/L, about 300 g/L, about 250 g/L, about 50 g/L, about 10 g/L, about 200 g/L, about 150 g/L, or about 100 g/L.

In certain embodiments, the composition (or the first preservation reagent, or the second preservation reagent) comprises one or more saccharides (e.g., sucrose) at a concentration ranging from about 0.1 M (mol/L) to about 2 M, from about 0.1 M to about 1.5 M, from about 0.1 M to about 1.2 M, from about 0.1 M to about 1.1 M, from about 0.1 M to about 1 M, from about 0.1 M to about 0.9 M, from about 0.1 M to about 0.8 M, from about 0.1 M to about 0.7 M, from about 0.1 M to about 0.6 M, from about 0.1 M to about 0.5 M, from about 0.1 M to about 0.4 M, from about 0.1 M to about 0.3 M, from about 0.2 M to about 1 M, from about 0.3 M to about 1 M, from about 0.4 M to about 1 M, from about 0.5 M to about 1 M, from about 0.6 M to about 1 M, from about 0.2 M to about 0.9 M, from about 0.2 M to about 0.8 M, from about 0.2 M to about 0.7 M, from about 0.2 M to about 0.6 M, from about 0.2 M to about 0.5 M, from about 0.2 M to about 0.4 M, from about 0.3 M to about 0.9 M, from about 0.3 M to about 0.8 M, from about 0.3 M to about 0.7 M, from about 0.3 M to about 0.6 M, from about 0.3 M to about 0.5 M, from about 0.4 M to about 0.9 M, from about 0.4 M to about 0.8 M, from about 0.4 M to about 0.7 M, from about 0.4 M to about 0.6 M, from about 0.5 M to about 0.8 M, from about 0.5 M to about 0.7 M, about 0.3 M, about 0.45 M, about 0.6 M, or about 0.75 M.

In certain embodiments, the preservation composition comprises potassium dihydrogen phosphate or sodium dihydrogen phosphate at a concentration ranging from about 30 mg/L to about 300 mg/L, from about 30 mg/L to about 250 mg/L, from about 30 mg/L to about 200 mg/L, from about 30 mg/L to about 150 mg/L, from about 40 mg/L to about 300 mg/L, from about 40 mg/L to about 250 mg/L, from about 40 mg/L to about 200 mg/L, from about 40 mg/L to about 150 mg/L, from about 50 mg/L to about 300 mg/L, from about 50 mg/L to about 250 mg/L, from about 50 mg/L to about 200 mg/L, from about 50 mg/L to about 150 mg/L, from about 70 mg/L to about 300 mg/L, from about 70 mg/L to about 250 mg/L, from about 70 mg/L to about 200 mg/L, from about 70 mg/L to about 180 mg/L, from about 70 mg/L to about 150 mg/L, from about 70 mg/L to about 140 mg/L, from about 70 mg/L to about 130 mg/L, from about 70 mg/L to about 120 mg/L, from about 70 mg/L to about 110 mg/L, from about 90 mg/L to about 300 mg/L, from about 90 mg/L to about 250 mg/L, from about 90 mg/L to about 200 mg/L, from about 90 mg/L to about 150 mg/L, from about 100 mg/L to about 300 mg/L, from about 100 mg/L to about 250 mg/L, from about 100 mg/L to about 200 mg/L, from about 100 mg/L to about 150 mg/L, from about 110 mg/L to about 150 mg/L, from about 120 mg/L to about 150 mg/L, about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, about 150 mg/L, or about 160 mg/L.

In certain embodiments, the preservation composition comprises potassium dihydrogen phosphate or sodium dihydrogen phosphate at a concentration ranging from about 0.3 mM to about 2 mM, from about 0.3 mM to about 1.9 mM, from about 0.3 mM to about 1.8 mM, from about 0.3 mM to about 1.7 mM, from about 0.3 mM to about 1.6 mM, from about 0.3 mM to about 1.5 mM, from about 0.3 mM to about 1.4 mM, from about 0.3 mM to about 1.3 mM, from about 0.3 mM to about 1.2 mM, from about 0.3 mM to about 1.1 mM, from about 0.3 mM to about 1 mM, from about 0.4 mM to about 2 mM, from about 0.4 mM to about 1.9 mM, from about 0.4 mM to about 1.8 mM, from about 0.4 mM to about 1.7 mM, from about 0.4 mM to about 1.6 mM, from about 0.4 mM to about 1.5 mM, from about 0.4 mM to about 1.4 mM, from about 0.4 mM to about 1.3 mM, from about 0.4 mM to about 1.2 mM, from about 0.4 mM to about 1.1 mM, from about 0.4 mM to about 1 mM, from about 0.5 mM to about 2 mM, from about 0.5 mM to about 1.9 mM, from about 0.5 mM to about 1.8 mM, from about 0.5 mM to about 1.7 mM, from about 0.5 mM to about 1.6 mM, from about 0.5 mM to about 1.5 mM, from about 0.5 mM to about 1.4 mM, from about 0.5 mM to about 1.3 mM, from about 0.5 mM to about 1.2 mM, from about 0.5 mM to about 1.1 mM, from about 0.5 mM to about 1 mM, from about 0.6 mM to about 2 mM, from about 0.6 mM to about 1.9 mM, from about 0.6 mM to about 1.8 mM, from about 0.6 mM to about 1.7 mM, from about 0.6 mM to about 1.6 mM, from about 0.6 mM to about 1.5 mM, from about 0.6 mM to about 1.4 mM, from about 0.6 mM to about 1.3 mM, from about 0.6 mM to about 1.2 mM, from about 0.6 mM to about 1.1 mM, from about 0.6 mM to about 1 mM, from about 0.7 mM to about 2 mM, from about 0.7 mM to about 1.9 mM, from about 0.7 mM to about 1.8 mM, from about 0.7 mM to about 1.7 mM, from about 0.7 mM to about 1.6 mM, from about 0.7 mM to about 1.5 mM, from about 0.7 mM to about 1.4 mM, from about 0.7 mM to about 1.3 mM, from about 0.7 mM to about 1.2 mM, from about 0.7 mM to about 1.1 mM, from about 0.7 mM to about 1 mM, from about 0.8 mM to about 2 mM, from about 0.8 mM to about 1.9 mM, from about 0.8 mM to about 1.8 mM, from about 0.8 mM to about 1.7 mM, from about 0.8 mM to about 1.6 mM, from about 0.8 mM to about 1.5 mM, from about 0.8 mM to about 1.4 mM, from about 0.8 mM to about 1.3 mM, from about 0.8 mM to about 1.2 mM, from about 0.8 mM to about 1.1 mM, from about 0.8 mM to about 1 mM, about 0.5 mM, about 0.6 mM, about 0.7 mM, about 0.8 mM, about 0.9 mM, about 1 mM, or about 1.1 mM.

In certain embodiments, the first preservation reagent comprises potassium dihydrogen phosphate or sodium dihydrogen phosphate at a concentration ranging from about 50 mg/L to about 300 mg/L, from about 50 mg/L to about 250 mg/L, from about 50 mg/L to about 200 mg/L, from about 50 mg/L to about 150 mg/L, from about 70 mg/L to about 300 mg/L, from about 70 mg/L to about 250 mg/L, from about 70 mg/L to about 200 mg/L, from about 70 mg/L to about 150 mg/L, from about 90 mg/L to about 300 mg/L, from about 90 mg/L to about 250 mg/L, from about 90 mg/L to about 200 mg/L, from about 90 mg/L to about 150 mg/L, from about 100 mg/L to about 300 mg/L, from about 100 mg/L to about 250 mg/L, from about 100 mg/L to about 200 mg/L, from about 100 mg/L to about 150 mg/L, from about 110 mg/L to about 150 mg/L, from about 120 mg/L to about 150 mg/L, about 100 mg/L, about 110 mg/L, about 120 mg/L, about 130 mg/L, about 140 mg/L, about 145 mg/L, about 144 mg/L, about 160 mg/L, about 170 mg/L, about 180 mg/L, or about 150 mg/L.

In certain embodiments, the first preservation reagent comprises potassium dihydrogen phosphate or sodium dihydrogen phosphate at a concentration ranging from about 0.5 mM to about 2 mM, from about 0.5 mM to about 1.9 mM, from about 0.5 mM to about 1.8 mM, from about 0.5 mM to about 1.7 mM, from about 0.5 mM to about 1.6 mM, from about 0.5 mM to about 1.5 mM, from about 0.5 mM to about 1.4 mM, from about 0.5 mM to about 1.3 mM, from about 0.5 mM to about 1.2 mM, from about 0.5 mM to about 1.1 mM, from about 0.5 mM to about 1 mM, from about 0.6 mM to about 2 mM, from about 0.6 mM to about 1.9 mM, from about 0.6 mM to about 1.8 mM, from about 0.6 mM to about 1.7 mM, from about 0.6 mM to about 1.6 mM, from about 0.6 mM to about 1.5 mM, from about 0.6 mM to about 1.4 mM, from about 0.6 mM to about 1.3 mM, from about 0.6 mM to about 1.2 mM, from about 0.6 mM to about 1.1 mM, from about 0.6 mM to about 1 mM, from about 0.7 mM to about 2 mM, from about 0.7 mM to about 1.9 mM, from about 0.7 mM to about 1.8 mM, from about 0.7 mM to about 1.7 mM, from about 0.7 mM to about 1.6 mM, from about 0.7 mM to about 1.5 mM, from about 0.7 mM to about 1.4 mM, from about 0.7 mM to about 1.3 mM, from about 0.7 mM to about 1.2 mM, from about 0.7 mM to about 1.1 mM, from about 0.7 mM to about 1 mM, from about 0.8 mM to about 2 mM, from about 0.8 mM to about 1.9 mM, from about 0.8 mM to about 1.8 mM, from about 0.8 mM to about 1.7 mM, from about 0.8 mM to about 1.6 mM, from about 0.8 mM to about 1.5 mM, from about 0.8 mM to about 1.4 mM, from about 0.8 mM to about 1.3 mM, from about 0.8 mM to about 1.2 mM, from about 0.8 mM to about 1.1 mM, from about 0.8 mM to about 1 mM, about 0.7 mM, about 0.8 mM, about 0.9 mM, about 1 mM or about 1.1 mM.

In certain embodiments, the second preservation reagent comprises potassium dihydrogen phosphate or sodium dihydrogen phosphate at a concentration ranging from about 10 mg/L to about 300 mg/L, from about 10 mg/L to about 250 mg/L, from about 10 mg/L to about 200 mg/L, from about 10 mg/L to about 150 mg/L, from about 20 mg/L to about 300 mg/L, from about 20 mg/L to about 250 mg/L, from about 20 mg/L to about 200 mg/L, from about 20 mg/L to about 150 mg/L, from about 30 mg/L to about 300 mg/L, from about 30 mg/L to about 250 mg/L, from about 30 mg/L to about 200 mg/L, from about 30 mg/L to about 150 mg/L, from about 40 mg/L to about 300 mg/L, from about 40 mg/L to about 250 mg/L, from about 40 mg/L to about 200 mg/L, from about 40 mg/L to about 150 mg/L, from about 50 mg/L to about 300 mg/L, from about 50 mg/L to about 250 mg/L, from about 50 mg/L to about 200 mg/L, from about 50 mg/L to about 150 mg/L, from about 70 mg/L to about 300 mg/L, from about 70 mg/L to about 250 mg/L, from about 70 mg/L to about 200 mg/L, from about 70 mg/L to about 150 mg/L, from about 90 mg/L to about 300 mg/L, from about 90 mg/L to about 250 mg/L, from about 90 mg/L to about 200 mg/L, from about 90 mg/L to about 150 mg/L, from about 100 mg/L to about 300 mg/L, from about 100 mg/L to about 250 mg/L, from about 100 mg/L to about 200 mg/L, from about 100 mg/L to about 150 mg/L, from about 110 mg/L to about 150 mg/L, from about 120 mg/L to about 150 mg/L, about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, about 145 mg/L, about 144 mg/L, about 160 mg/L, about 170 mg/L, about 180 mg/L, or about 150 mg/L.

In certain embodiments, the second preservation reagent comprises potassium dihydrogen phosphate or sodium dihydrogen phosphate at a concentration ranging from about 0.05 mM to about 2 mM, from about 0.05 mM to about 1.9 mM, from about 0.05 mM to about 1.8 mM, from about 0.05 mM to about 1.7 mM, from about 0.05 mM to about 1.6 mM, from about 0.05 mM to about 1.5 mM, from about 0.05 mM to about 1.4 mM, from about 0.05 mM to about 1.3 mM, from about 0.05 mM to about 1.2 mM, from about 0.05 mM to about 1.1 mM, from about 0.05 mM to about 1 mM, from about 0.08 mM to about 2 mM, from about 0.08 mM to about 1.9 mM, from about 0.08 mM to about 1.8 mM, from about 0.08 mM to about 1.7 mM, from about 0.08 mM to about 1.6 mM, from about 0.08 mM to about 1.5 mM, from about 0.08 mM to about 1.4 mM, from about 0.08 mM to about 1.3 mM, from about 0.08 mM to about 1.2 mM, from about 0.08 mM to about 1.1 mM, from about 0.08 mM to about 1 mM, from about 0.1 mM to about 2 mM, from about 0.1 mM to about 1.9 mM, from about 0.1 mM to about 1.8 mM, from about 0.1 mM to about 1.7 mM, from about 0.1 mM to about 1.6 mM, from about 0.1 mM to about 1.5 mM, from about 0.1 mM to about 1.4 mM, from about 0.1 mM to about 1.3 mM, from about 0.1 mM to about 1.2 mM, from about 0.1 mM to about 1.1 mM, from about 0.1 mM to about 1 mM, from about 0.12 mM to about 2 mM, from about 0.12 mM to about 1.9 mM, from about 0.12 mM to about 1.8 mM, from about 0.12 mM to about 1.7 mM, from about 0.12 mM to about 1.6 mM, from about 0.12 mM to about 1.5 mM, from about 0.12 mM to about 1.4 mM, from about 0.12 mM to about 1.3 mM, from about 0.12 mM to about 1.2 mM, from about 0.12 mM to about 1.1 mM, from about 0.12 mM to about 1 mM, from about 0.14 mM to about 2 mM, from about 0.14 mM to about 1.9 mM, from about 0.14 mM to about 1.8 mM, from about 0.14 mM to about 1.7 mM, from about 0.14 mM to about 1.6 mM, from about 0.14 mM to about 1.5 mM, from about 0.14 mM to about 1.4 mM, from about 0.14 mM to about 1.3 mM, from about 0.14 mM to about 1.2 mM, from about 0.14 mM to about 1.1 mM, from about 0.14 mM to about 1 mM, from about 0.5 mM to about 2 mM, from about 0.5 mM to about 1.9 mM, from about 0.5 mM to about 1.8 mM, from about 0.5 mM to about 1.7 mM, from about 0.5 mM to about 1.6 mM, from about 0.5 mM to about 1.5 mM, from about 0.5 mM to about 1.4 mM, from about 0.5 mM to about 1.3 mM, from about 0.5 mM to about 1.2 mM, from about 0.5 mM to about 1.1 mM, from about 0.5 mM to about 1 mM, from about 0.6 mM to about 2 mM, from about 0.6 mM to about 1.9 mM, from about 0.6 mM to about 1.8 mM, from about 0.6 mM to about 1.7 mM, from about 0.6 mM to about 1.6 mM, from about 0.6 mM to about 1.5 mM, from about 0.6 mM to about 1.4 mM, from about 0.6 mM to about 1.3 mM, from about 0.6 mM to about 1.2 mM, from about 0.6 mM to about 1.1 mM, from about 0.6 mM to about 1 mM, from about 0.7 mM to about 2 mM, from about 0.7 mM to about 1.9 mM, from about 0.7 mM to about 1.8 mM, from about 0.7 mM to about 1.7 mM, from about 0.7 mM to about 1.6 mM, from about 0.7 mM to about 1.5 mM, from about 0.7 mM to about 1.4 mM, from about 0.7 mM to about 1.3 mM, from about 0.7 mM to about 1.2 mM, from about 0.7 mM to about 1.1 mM, from about 0.7 mM to about 1 mM, from about 0.8 mM to about 2 mM, from about 0.8 mM to about 1.9 mM, from about 0.8 mM to about 1.8 mM, from about 0.8 mM to about 1.7 mM, from about 0.8 mM to about 1.6 mM, from about 0.8 mM to about 1.5 mM, from about 0.8 mM to about 1.4 mM, from about 0.8 mM to about 1.3 mM, from about 0.8 mM to about 1.2 mM, from about 0.8 mM to about 1.1 mM, from about 0.8 mM to about 1 mM, about 0.7 mM, about 0.8 mM, about 0.9 mM, about 1 mM or about 1.1 mM.

In certain embodiments, the preservation composition comprises disodium hydrogen phosphate heptahydrate or dipotassium hydrogen phosphate heptahydrate at a concentration ranging from about 200 mg/L to about 1000 mg/L, from about 200 mg/L to about 950 mg/L, from about 200 mg/L to about 900 mg/L, from about 200 mg/L to about 850 mg/L, from about 200 mg/L to about 800 mg/L, from about 200 mg/L to about 750 mg/L, from about 200 mg/L to about 700 mg/L, from about 200 mg/L to about 600 mg/L, from about 200 mg/L to about 500 mg/L, from about 300 mg/L to about 1000 mg/L, from about 300 mg/L to about 950 mg/L, from about 300 mg/L to about 900 mg/L, from about 300 mg/L to about 850 mg/L, from about 300 mg/L to about 800 mg/L, from about 300 mg/L to about 750 mg/L, from about 300 mg/L to about 700 mg/L, from about 300 mg/L to about 600 mg/L, from about 300 mg/L to about 500 mg/L, from about 400 mg/L to about 1000 mg/L, from about 400 mg/L to about 950 mg/L, from about 400 mg/L to about 900 mg/L, from about 400 mg/L to about 850 mg/L, from about 400 mg/L to about 800 mg/L, from about 400 mg/L to about 750 mg/L, from about 400 mg/L to about 700 mg/L, from about 400 mg/L to about 600 mg/L, from about 400 mg/L to about 500 mg/L, from about 500 mg/L to about 1000 mg/L, from about 500 mg/L to about 950 mg/L, from about 500 mg/L to about 900 mg/L, from about 500 mg/L to about 850 mg/L, from about 500 mg/L to about 800 mg/L, from about 500 mg/L to about 750 mg/L, from about 500 mg/L to about 700 mg/L, about 200 mg/L, about 300 mg/L, about 350 mg/L, about 400 mg/L, about 450 mg/L, about 500 mg/L, about 550 mg/L, about 600 mg/L, about 700 mg/L, about 800 mg/L, about 900 mg/L, or about 1000 mg/L.

In certain embodiments, the preservation composition comprises disodium hydrogen phosphate or dipotassium hydrogen phosphate at a concentration ranging from about 1 mM to about 5 mM, from about 1 mM to about 4.5 mM, from about 1 mM to about 4 mM, from about 1 mM to about 3.5 mM, from about 1 mM to about 3 mM, from about 1 mM to about 2.5 mM, from about 1 mM to about 2 mM, from about 1.2 mM to about 5 mM, from about 1.2 mM to about 4.5 mM, from about 1.2 mM to about 4 mM, from about 1.2 mM to about 3.5 mM, from about 1.2 mM to about 3 mM, from about 1.2 mM to about 2.5 mM, from about 1.2 mM to about 2 mM, from about 1.5 mM to about 5 mM, from about 1.5 mM to about 4.5 mM, from about 1.5 mM to about 4 mM, from about 1.5 mM to about 3.5 mM, from about 1.5 mM to about 3 mM, from about 1.5 mM to about 2.5 mM, from about 1.5 mM to about 2 mM, from about 1.6 mM to about 5 mM, from about 1.6 mM to about 4.5 mM, from about 1.6 mM to about 4 mM, from about 1.6 mM to about 3.5 mM, from about 1.6 mM to about 3 mM, from about 1.6 mM to about 2.5 mM, from about 1.6 mM to about 2 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, or about 3 mM.

In certain embodiments, the first preservation agent comprises disodium hydrogen phosphate heptahydrate or dipotassium hydrogen phosphate heptahydrate at a concentration ranging from about 300 mg/L to about 1000 mg/L, from about 300 mg/L to about 950 mg/L, from about 300 mg/L to about 900 mg/L, from about 300 mg/L to about 850 mg/L, from about 300 mg/L to about 800 mg/L, from about 300 mg/L to about 750 mg/L, from about 300 mg/L to about 700 mg/L, from about 300 mg/L to about 600 mg/L, from about 300 mg/L to about 500 mg/L, from about 400 mg/L to about 1000 mg/L, from about 400 mg/L to about 950 mg/L, from about 400 mg/L to about 900 mg/L, from about 400 mg/L to about 850 mg/L, from about 400 mg/L to about 800 mg/L, from about 400 mg/L to about 750 mg/L, from about 400 mg/L to about 700 mg/L, from about 400 mg/L to about 600 mg/L, from about 400 mg/L to about 500 mg/L, from about 500 mg/L to about 1000 mg/L, from about 500 mg/L to about 950 mg/L, from about 500 mg/L to about 900 mg/L, from about 500 mg/L to about 850 mg/L, from about 500 mg/L to about 800 mg/L, from about 500 mg/L to about 750 mg/L, from about 500 mg/L to about 700 mg/L, about 400 mg/L, about 450 mg/L, about 500 mg/L, about 550 mg/L, about 600 mg/L, about 700 mg/L, about 795 mg/L, about 800 mg/L, about 850 mg/L, about 900 mg/L, or about 1000 mg/L.

In certain embodiments, the first preservation agent comprises disodium hydrogen phosphate or dipotassium hydrogen phosphate at a concentration ranging from about 1.5 mM to about 5 mM, from about 1.5 mM to about 4.5 mM, from about 1.5 mM to about 4 mM, from about 1.5 mM to about 3.5 mM, from about 1.5 mM to about 3 mM, from about 1.5 mM to about 2.5 mM, from about 1.5 mM to about 2 mM, from about 1.6 mM to about 5 mM, from about 1.6 mM to about 4.5 mM, from about 1.6 mM to about 4 mM, from about 1.6 mM to about 3.5 mM, from about 1.6 mM to about 3 mM, from about 1.6 mM to about 2.5 mM, from about 1.6 mM to about 2 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, about 3.7 mM, about 3.8 mM, about 3.9 mM, or about 4 mM.

In certain embodiments, the second preservation agent comprises disodium hydrogen phosphate heptahydrate or dipotassium hydrogen phosphate heptahydrate at a concentration ranging from about 100 mg/L to about 1000 mg/L, from about 100 mg/L to about 950 mg/L, from about 100 mg/L to about 900 mg/L, from about 100 mg/L to about 850 mg/L, from about 100 mg/L to about 800 mg/L, from about 100 mg/L to about 750 mg/L, from about 100 mg/L to about 700 mg/L, from about 100 mg/L to about 600 mg/L, from about 100 mg/L to about 500 mg/L, from about 150 mg/L to about 1000 mg/L, from about 150 mg/L to about 950 mg/L, from about 150 mg/L to about 900 mg/L, from about 150 mg/L to about 850 mg/L, from about 150 mg/L to about 800 mg/L, from about 150 mg/L to about 750 mg/L, from about 150 mg/L to about 700 mg/L, from about 150 mg/L to about 600 mg/L, from about 150 mg/L to about 500 mg/L, from about 200 mg/L to about 1000 mg/L, from about 200 mg/L to about 950 mg/L, from about 200 mg/L to about 900 mg/L, from about 200 mg/L to about 850 mg/L, from about 200 mg/L to about 800 mg/L, from about 200 mg/L to about 750 mg/L, from about 200 mg/L to about 700 mg/L, from about 200 mg/L to about 600 mg/L, from about 200 mg/L to about 500 mg/L, from about 300 mg/L to about 1000 mg/L, from about 300 mg/L to about 950 mg/L, from about 300 mg/L to about 900 mg/L, from about 300 mg/L to about 850 mg/L, from about 300 mg/L to about 800 mg/L, from about 300 mg/L to about 750 mg/L, from about 300 mg/L to about 700 mg/L, from about 300 mg/L to about 600 mg/L, from about 300 mg/L to about 500 mg/L, from about 400 mg/L to about 1000 mg/L, from about 400 mg/L to about 950 mg/L, from about 400 mg/L to about 900 mg/L, from about 400 mg/L to about 850 mg/L, from about 400 mg/L to about 800 mg/L, from about 400 mg/L to about 750 mg/L, from about 400 mg/L to about 700 mg/L, from about 400 mg/L to about 600 mg/L, from about 400 mg/L to about 500 mg/L, from about 500 mg/L to about 1000 mg/L, from about 500 mg/L to about 950 mg/L, from about 500 mg/L to about 900 mg/L, from about 500 mg/L to about 850 mg/L, from about 500 mg/L to about 800 mg/L, from about 500 mg/L to about 750 mg/L, from about 500 mg/L to about 700 mg/L, about 100 mg/L, about 150 mg/L, about 167 mg/L, about 170 mg/L, about 200 mg/L, about 300 mg/L, about 350 mg/L, about 400 mg/L, about 450 mg/L, about 500 mg/L, about 550 mg/L, about 600 mg/L, about 700 mg/L, about 795 mg/L, about 800 mg/L, about 900 mg/L, or about 1000 mg/L.

In certain embodiments, the second preservation agent comprises disodium hydrogen phosphate or dipotassium hydrogen phosphate at a concentration ranging from about 0.1 mM to about 5 mM, from about 0.1 mM to about 4.5 mM, from about 0.1 mM to about 4 mM, from about 0.1 mM to about 3.5 mM, from about 0.1 mM to about 3 mM, from about 0.1 mM to about 2.5 mM, from about 0.1 mM to about 2 mM, from about 0.2 mM to about 5 mM, from about 0.2 mM to about 4.5 mM, from about 0.2 mM to about 4 mM, from about 0.2 mM to about 3.5 mM, from about 0.2 mM to about 3 mM, from about 0.2 mM to about 2.5 mM, from about 0.2 mM to about 2 mM, from about 0.3 mM to about 5 mM, from about 0.3 mM to about 4.5 mM, from about 0.3 mM to about 4 mM, from about 0.3 mM to about 3.5 mM, from about 0.3 mM to about 3 mM, from about 0.3 mM to about 2.5 mM, from about 0.3 mM to about 2 mM, from about 0.4 mM to about 5 mM, from about 0.4 mM to about 4.5 mM, from about 0.4 mM to about 4 mM, from about 0.4 mM to about 3.5 mM, from about 0.4 mM to about 3 mM, from about 0.4 mM to about 2.5 mM, from about 0.4 mM to about 2 mM, from about 0.5 mM to about 5 mM, from about 0.5 mM to about 4.5 mM, from about 0.5 mM to about 4 mM, from about 0.5 mM to about 3.5 mM, from about 0.5 mM to about 3 mM, from about 0.5 mM to about 2.5 mM, from about 0.5 mM to about 2 mM, from about 1 mM to about 5 mM, from about 1 mM to about 4.5 mM, from about 1 mM to about 4 mM, from about 1 mM to about 3.5 mM, from about 1 mM to about 3 mM, from about 1 mM to about 2.5 mM, from about 1 mM to about 2 mM, from about 1.2 mM to about 5 mM, from about 1.2 mM to about 4.5 mM, from about 1.2 mM to about 4 mM, from about 1.2 mM to about 3.5 mM, from about 1.2 mM to about 3 mM, from about 1.2 mM to about 2.5 mM, from about 1.2 mM to about 2 mM, from about 1.5 mM to about 5 mM, from about 1.5 mM to about 4.5 mM, from about 1.5 mM to about 4 mM, from about 1.5 mM to about 3.5 mM, from about 1.5 mM to about 3 mM, from about 1.5 mM to about 2.5 mM, from about 1.5 mM to about 2 mM, from about 1.6 mM to about 5 mM, from about 1.6 mM to about 4.5 mM, from about 1.6 mM to about 4 mM, from about 1.6 mM to about 3.5 mM, from about 1.6 mM to about 3 mM, from about 1.6 mM to about 2.5 mM, from about 1.6 mM to about 2 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, or about 3 mM.

In certain embodiments, the preservation composition comprises sodium chloride at a concentration ranging from about 2 g/L to about 20 g/L, from about 2 g/L to about 15 g/L, from about 2 g/L to about 12 g/L, from about 2 g/L to about 10 g/L, from about 2 g/L to about 9 g/L, from about 3 g/L to about 20 g/L, from about 3 g/L to about 15 g/L, from about 3 g/L to about 12 g/L, from about 3 g/L to about 10 g/L, from about 3 g/L to about 9 g/L, from about 4 g/L to about 20 g/L, from about 4 g/L to about 15 g/L, from about 4 g/L to about 12 g/L, from about 4 g/L to about 10 g/L, from about 4 g/L to about 9 g/L, from about 5 g/L to about 20 g/L, from about 5 g/L to about 15 g/L, from about 5 g/L to about 12 g/L, from about 5 g/L to about 10 g/L, from about 5 g/L to about 9 g/L, from about 5.5 g/L to about 20 g/L, from about 5.5 g/L to about 15 g/L, from about 5.5 g/L to about 12 g/L, from about 5.5 g/L to about 10 g/L, from about 5.5 g/L to about 9 g/L, from about 6 g/L to about 20 g/L, from about 6 g/L to about 15 g/L, from about 6 g/L to about 12 g/L, from about 6 g/L to about 10 g/L, from about 6 g/L to about 9 g/L, from about 7 g/L to about 20 g/L, from about 7 g/L to about 15 g/L, from about 7 g/L to about 12 g/L, from about 7 g/L to about 10 g/L, from about 7 g/L to about 9 g/L, about 2 g/L, about 3 g/L, about 4 g/L, about 5 g/L, about 5.5 g/L, about 6 g/L, about 7 g/L, about 8 g/L, about 8.5 g/L, about 9 g/L, or about 9.5 g/L.

In certain embodiments, the first preservation agent comprises sodium chloride at a concentration ranging from about 4 g/L to about 20 g/L, from about 4 g/L to about 15 g/L, from about 4 g/L to about 12 g/L, from about 4 g/L to about 10 g/L, from about 4 g/L to about 9 g/L, from about 5 g/L to about 20 g/L, from about 5 g/L to about 15 g/L, from about 5 g/L to about 12 g/L, from about 5 g/L to about 10 g/L, from about 5 g/L to about 9 g/L, from about 5.5 g/L to about 20 g/L, from about 5.5 g/L to about 15 g/L, from about 5.5 g/L to about 12 g/L, from about 5.5 g/L to about 10 g/L, from about 5.5 g/L to about 9 g/L, from about 6 g/L to about 20 g/L, from about 6 g/L to about 15 g/L, from about 6 g/L to about 12 g/L, from about 6 g/L to about 10 g/L, from about 6 g/L to about 9 g/L, from about 7 g/L to about 20 g/L, from about 7 g/L to about 15 g/L, from about 7 g/L to about 12 g/L, from about 7 g/L to about 10 g/L, from about 7 g/L to about 9 g/L, about 2 g/L, about 3 g/L, about 4 g/L, about 5 g/L, about 5.5 g/L, about 6 g/L, about 7 g/L, about 8 g/L, about 8.5 g/L, about 9 g/L, or about 9.5 g/L.

In certain embodiments, the second preservation agent comprises sodium chloride at a concentration ranging from about 0.5 g/L to about 20 g/L, from about 0.5 g/L to about 15 g/L, from about 0.5 g/L to about 12 g/L, from about 0.5 g/L to about 10 g/L, from about 0.5 g/L to about 9 g/L, from about 1 g/L to about 20 g/L, from about 1 g/L to about 15 g/L, from about 1 g/L to about 12 g/L, from about 1 g/L to about 10 g/L, from about 1 g/L to about 9 g/L, from about 1.5 g/L to about 20 g/L, from about 1.5 g/L to about 15 g/L, from about 1.5 g/L to about 12 g/L, from about 1.5 g/L to about 10 g/L, from about 1.5 g/L to about 9 g/L, from about 2 g/L to about 20 g/L, from about 2 g/L to about 15 g/L, from about 2 g/L to about 12 g/L, from about 2 g/L to about 10 g/L, from about 2 g/L to about 9 g/L, from about 3 g/L to about 20 g/L, from about 3 g/L to about 15 g/L, from about 3 g/L to about 12 g/L, from about 3 g/L to about 10 g/L, from about 3 g/L to about 9 g/L, from about 4 g/L to about 20 g/L, from about 4 g/L to about 15 g/L, from about 4 g/L to about 12 g/L, from about 4 g/L to about 10 g/L, from about 4 g/L to about 9 g/L, from about 5 g/L to about 20 g/L, from about 5 g/L to about 15 g/L, from about 5 g/L to about 12 g/L, from about 5 g/L to about 10 g/L, from about 5 g/L to about 9 g/L, from about 5.5 g/L to about 20 g/L, from about 5.5 g/L to about 15 g/L, from about 5.5 g/L to about 12 g/L, from about 5.5 g/L to about 10 g/L, from about 5.5 g/L to about 9 g/L, from about 6 g/L to about 20 g/L, from about 6 g/L to about 15 g/L, from about 6 g/L to about 12 g/L, from about 6 g/L to about 10 g/L, from about 6 g/L to about 9 g/L, from about 7 g/L to about 20 g/L, from about 7 g/L to about 15 g/L, from about 7 g/L to about 12 g/L, from about 7 g/L to about 10 g/L, from about 7 g/L to about 9 g/L, about 1.5 g/L, about 1.8 g/L, about 2 g/L, about 3 g/L, about 4 g/L, about 5 g/L, about 5.5 g/L, about 6 g/L, about 7 g/L, about 8 g/L, about 8.5 g/L, about 9 g/L, or about 9.5 g/L.

In certain embodiments, the preservation composition comprises one or more macromolecules (e.g., albumin such as human albumin) at a concentration ranging from about 10 g/L to about 500 g/L, from about 10 g/L to about 400 g/L, from about 10 g/L to about 350 g/L, from about 10 g/L to about 300 g/L, from about 10 g/L to about 250 g/L, from about 10 g/L to about 240 g/L, from about 10 g/L to about 200 g/L, from about 10 g/L to about 150 g/L, from about 10 g/L to about 120 g/L, from about 10 g/L to about 100 g/L, from about 20 g/L to about 500 g/L, from about 20 g/L to about 400 g/L, from about 20 g/L to about 350 g/L, from about 20 g/L to about 300 g/L, from about 20 g/L to about 250 g/L, from about 20 g/L to about 240 g/L, from about 20 g/L to about 200 g/L, from about 20 g/L to about 150 g/L, from about 20 g/L to about 120 g/L, from about 20 g/L to about 100 g/L, from about 30 g/L to about 500 g/L, from about 30 g/L to about 400 g/L, from about 30 g/L to about 350 g/L, from about 30 g/L to about 300 g/L, from about 30 g/L to about 250 g/L, from about 30 g/L to about 240 g/L, from about 30 g/L to about 200 g/L, from about 30 g/L to about 150 g/L, from about 30 g/L to about 120 g/L, from about 30 g/L to about 100 g/L, from about 40 g/L to about 500 g/L, from about 40 g/L to about 400 g/L, from about 40 g/L to about 350 g/L, from about 40 g/L to about 300 g/L, from about 40 g/L to about 250 g/L, from about 40 g/L to about 240 g/L, from about 40 g/L to about 200 g/L, from about 40 g/L to about 150 g/L, from about 40 g/L to about 120 g/L, from about 40 g/L to about 100 g/L, from about 50 g/L to about 500 g/L, from about 50 g/L to about 400 g/L, from about 50 g/L to about 350 g/L, from about 50 g/L to about 300 g/L, from about 50 g/L to about 250 g/L, from about 50 g/L to about 240 g/L, from about 50 g/L to about 200 g/L, from about 50 g/L to about 150 g/L, from about 50 g/L to about 120 g/L, from about 50 g/L to about 100 g/L, from about 150 g/L to about 500 g/L, from about 150 g/L to about 400 g/L, from about 150 g/L to about 350 g/L, from about 150 g/L to about 300 g/L, from about 150 g/L to about 250 g/L, from about 150 g/L to about 240 g/L, from about 150 g/L to about 200 g/L, from about 200 g/L to about 500 g/L, from about 200 g/L to about 400 g/L, from about 200 g/L to about 350 g/L, from about 200 g/L to about 300 g/L, from about 200 g/L to about 250 g/L, from about 200 g/L to about 240 g/L, from about 100 g/L to about 500 g/L, from about 100 g/L to about 450 g/L, from about 100 g/L to about 400 g/L, from about 100 g/L to about 350 g/L, from about 100 g/L to about 300 g/L, from about 100 g/L to about 200 g/L, from about 100 g/L to about 150 g/L, from about 100 g/L to about 120 g/L, from about 100 g/L to about 250 g/L, from about 100 g/L to about 240 g/L, about 10 g/L, about 20 g/L, about 30 g/L, about 40 g/L, about 50 g/L, about 60 g/L, about 150 g/L, about 120 g/L, or about 100 g/L.

In certain embodiments, the second preservation agent comprises one or more macromolecules (e.g., albumin such as human albumin) at a concentration ranging from about 10 g/L to about 500 g/L, from about 30 g/L to about 500 g/L, from about 30 g/L to about 400 g/L, from about 30 g/L to about 350 g/L, from about 30 g/L to about 300 g/L, from about 30 g/L to about 250 g/L, from about 30 g/L to about 240 g/L, from about 30 g/L to about 200 g/L, from about 30 g/L to about 150 g/L, from about 30 g/L to about 120 g/L, from about 30 g/L to about 100 g/L, from about 40 g/L to about 500 g/L, from about 40 g/L to about 400 g/L, from about 40 g/L to about 350 g/L, from about 40 g/L to about 300 g/L, from about 40 g/L to about 250 g/L, from about 40 g/L to about 240 g/L, from about 40 g/L to about 200 g/L, from about 40 g/L to about 150 g/L, from about 40 g/L to about 120 g/L, from about 40 g/L to about 100 g/L, from about 50 g/L to about 500 g/L, from about 50 g/L to about 400 g/L, from about 50 g/L to about 350 g/L, from about 50 g/L to about 300 g/L, from about 50 g/L to about 250 g/L, from about 50 g/L to about 240 g/L, from about 50 g/L to about 200 g/L, from about 50 g/L to about 150 g/L, from about 50 g/L to about 120 g/L, from about 50 g/L to about 100 g/L, from about 150 g/L to about 500 g/L, from about 150 g/L to about 400 g/L, from about 150 g/L to about 350 g/L, from about 150 g/L to about 300 g/L, from about 150 g/L to about 250 g/L, from about 150 g/L to about 240 g/L, from about 150 g/L to about 200 g/L, from about 200 g/L to about 500 g/L, from about 200 g/L to about 400 g/L, from about 200 g/L to about 350 g/L, from about 200 g/L to about 300 g/L, from about 200 g/L to about 250 g/L, from about 200 g/L to about 240 g/L, from about 100 g/L to about 500 g/L, from about 100 g/L to about 450 g/L, from about 100 g/L to about 400 g/L, from about 100 g/L to about 350 g/L, from about 100 g/L to about 300 g/L, from about 100 g/L to about 200 g/L, from about 100 g/L to about 150 g/L, from about 100 g/L to about 120 g/L, from about 100 g/L to about 250 g/L, from about 100 g/L to about 240 g/L, about 400 g/L, about 350 g/L, about 300 g/L, about 250 g/L, about 240 g/L, about 50 g/L, about 60 g/L, about 10 g/L, about 200 g/L, about 150 g/L, about 120 g/L, or about 100 g/L.

In certain embodiments, the preservation composition comprises sodium caprylate or potassium caprylate at a concentration ranging from about 0.5 mM to about 30 mM, from about 0.5 mM to about 25 mM, from about 0.5 mM to about 20 mM, from about 0.5 mM to about 15 mM, from about 0.5 mM to about 10 mM, from about 0.5 mM to about 8 mM, from about 0.5 mM to about 5 mM, from about 1 mM to about 30 mM, from about 1 mM to about 25 mM, from about 1 mM to about 20 mM, from about 1 mM to about 15 mM, from about 1 mM to about 10 mM, from about 1 mM to about 8 mM, from about 1 mM to about 5 mM, from about 1.5 mM to about 30 mM, from about 1.5 mM to about 25 mM, from about 1.5 mM to about 20 mM, from about 1.5 mM to about 15 mM, from about 1.5 mM to about 10 mM, from about 1.5 mM to about 8 mM, from about 1.5 mM to about 5 mM, from about 1.6 mM to about 30 mM, from about 1.6 mM to about 25 mM, from about 1.6 mM to about 20 mM, from about 1.6 mM to about 15 mM, from about 1.6 mM to about 10 mM, from about 1.6 mM to about 8 mM, from about 1.6 mM to about 5 mM, from about 2 mM to about 30 mM, from about 2 mM to about 25 mM, from about 2 mM to about 20 mM, from about 2 mM to about 15 mM, from about 2 mM to about 10 mM, from about 2 mM to about 8 mM, from about 2 mM to about 5 mM, from about 3 mM to about 30 mM, from about 3 mM to about 25 mM, from about 3 mM to about 20 mM, from about 3 mM to about 15 mM, from about 3 mM to about 10 mM, from about 3 mM to about 8 mM, from about 3 mM to about 5 mM, from about 4 mM to about 30 mM, from about 4 mM to about 25 mM, from about 4 mM to about 20 mM, from about 4 mM to about 15 mM, from about 4 mM to about 10 mM, from about 4 mM to about 8 mM, from about 4 mM to about 5 mM, about 1.6 mM, about 4.8 mM, about 5 mM, about 9.6 mM, or about 10 mM.

In certain embodiments, the second preservation agent comprises sodium caprylate or potassium caprylate at a concentration ranging from about 0.5 mM to about 30 mM, from about 0.5 mM to about 25 mM, from about 0.5 mM to about 20 mM, from about 0.5 mM to about 15 mM, from about 0.5 mM to about 10 mM, from about 0.5 mM to about 8 mM, from about 0.5 mM to about 5 mM, from about 1 mM to about 30 mM, from about 1 mM to about 25 mM, from about 1 mM to about 20 mM, from about 1 mM to about 15 mM, from about 1 mM to about 10 mM, from about 1 mM to about 8 mM, from about 1 mM to about 5 mM, from about 2 mM to about 30 mM, from about 2 mM to about 25 mM, from about 2 mM to about 20 mM, from about 2 mM to about 15 mM, from about 2 mM to about 10 mM, from about 2 mM to about 8 mM, from about 2 mM to about 5 mM, from about 3 mM to about 30 mM, from about 3 mM to about 25 mM, from about 3 mM to about 20 mM, from about 3 mM to about 15 mM, from about 3 mM to about 10 mM, from about 3 mM to about 8 mM, from about 3 mM to about 5 mM, from about 4 mM to about 30 mM, from about 4 mM to about 25 mM, from about 4 mM to about 20 mM, from about 4 mM to about 15 mM, from about 4 mM to about 10 mM, from about 4 mM to about 8 mM, from about 4 mM to about 5 mM, about 4.8 mM, about 5 mM, about 9.6 mM, about 10 mM, about 20 mM, or about 19.2 mM.

In certain embodiments, the preservation composition comprises sodium N-acetyltryptophan or potassium N-acetyltryptophan at a concentration ranging from about 0.5 mM to about 30 mM, from about 0.5 mM to about 25 mM, from about 0.5 mM to about 20 mM, from about 0.5 mM to about 15 mM, from about 0.5 mM to about 10 mM, from about 0.5 mM to about 8 mM, from about 0.5 mM to about 5 mM, from about 1 mM to about 30 mM, from about 1 mM to about 25 mM, from about 1 mM to about 20 mM, from about 1 mM to about 15 mM, from about 1 mM to about 10 mM, from about 1 mM to about 8 mM, from about 1 mM to about 5 mM, from about 1.5 mM to about 30 mM, from about 1.5 mM to about 25 mM, from about 1.5 mM to about 20 mM, from about 1.5 mM to about 15 mM, from about 1.5 mM to about 10 mM, from about 1.5 mM to about 8 mM, from about 1.5 mM to about 5 mM, from about 1.6 mM to about 30 mM, from about 1.6 mM to about 25 mM, from about 1.6 mM to about 20 mM, from about 1.6 mM to about 15 mM, from about 1.6 mM to about 10 mM, from about 1.6 mM to about 8 mM, from about 1.6 mM to about 5 mM, from about 2 mM to about 30 mM, from about 2 mM to about 25 mM, from about 2 mM to about 20 mM, from about 2 mM to about 15 mM, from about 2 mM to about 10 mM, from about 2 mM to about 8 mM, from about 2 mM to about 5 mM, from about 3 mM to about 30 mM, from about 3 mM to about 25 mM, from about 3 mM to about 20 mM, from about 3 mM to about 15 mM, from about 3 mM to about 10 mM, from about 3 mM to about 8 mM, from about 3 mM to about 5 mM, from about 4 mM to about 30 mM, from about 4 mM to about 25 mM, from about 4 mM to about 20 mM, from about 4 mM to about 15 mM, from about 4 mM to about 10 mM, from about 4 mM to about 8 mM, from about 4 mM to about 5 mM, about 1.6 mM, about 4.8 mM, about 5 mM, about 9.6 mM, or about 10 mM.

In certain embodiments, the second preservation agent comprises sodium caprylate or potassium caprylate at a concentration ranging from about 0.5 mM to about 30 mM, from about 0.5 mM to about 25 mM, from about 0.5 mM to about 20 mM, from about 0.5 mM to about 15 mM, from about 0.5 mM to about 10 mM, from about 0.5 mM to about 8 mM, from about 0.5 mM to about 5 mM, from about 1 mM to about 30 mM, from about 1 mM to about 25 mM, from about 1 mM to about 20 mM, from about 1 mM to about 15 mM, from about 1 mM to about 10 mM, from about 1 mM to about 8 mM, from about 1 mM to about 5 mM, from about 2 mM to about 30 mM, from about 2 mM to about 25 mM, from about 2 mM to about 20 mM, from about 2 mM to about 15 mM, from about 2 mM to about 10 mM, from about 2 mM to about 8 mM, from about 2 mM to about 5 mM, from about 3 mM to about 30 mM, from about 3 mM to about 25 mM, from about 3 mM to about 20 mM, from about 3 mM to about 15 mM, from about 3 mM to about 10 mM, from about 3 mM to about 8 mM, from about 3 mM to about 5 mM, from about 4 mM to about 30 mM, from about 4 mM to about 25 mM, from about 4 mM to about 20 mM, from about 4 mM to about 15 mM, from about 4 mM to about 10 mM, from about 4 mM to about 8 mM, from about 4 mM to about 5 mM, about 4.8 mM, about 5 mM, about 9.6 mM, about 10 mM, about 20 mM, or about 19.2 mM.

The term “preservation” refers to the process of maintaining a biological material under conditions in which its biological activity is considerably reduced while it nonetheless remains viable and may resume essentially normal biological activity when taken out of the preservation state. Specific examples of preservation are cryopreservation and hypothermic preservation.

The term “preservation composition” relates to a composition (or a composition when diluted or dissolved) permitting the preservation of a biological material, such that the biological material retains its viability. A specific embodiment of a preservation composition is one for preserving biological material at low temperatures. In certain embodiments, the preservation composition is a preservation solution. Hypothermic preservation compositions and cryopreservation compositions are examples of preservation compositions.

The term “cryopreservation” refers to a process including at least one step of lowering the temperature of a biological material from a temperature that is above the freezing temperature of the biological material (or of a mixture of the biological material and a preservation composition) to a temperature that is below that freezing temperature. Cryopreservation encompasses freezing and lyophilization.

The terms “cryopreservation composition”, “cryopreservation medium”, or “freezing composition”, refer to a composition or medium (or a composition when diluted or dissolved) in which a biological material is immersed before cryopreservation or freezing, or to a composition or medium which can be used to treat the viral vectors or viral particles prior to freezing. In certain embodiments, a cryopreservation solution may be a freezing solution, a lyophilization solution and/or a mixture of such solutions. In certain embodiments, a cryopreservation composition is a cryopreservation solution. In certain embodiments, the cryopreservation composition refers to a composition or medium for storing or freezing viral vectors or viral particles at a temperature at or below about 8° C., at or below about 4° C., at or below about 0° C., at or below about −20° C., at or below about −70° C., at or below about −80° C., at or below about −135° C., at or below about −196° C., or in liquid nitrogen.

The term “hypothermic preservation” means preservation at a temperature below the physiological temperature but above freezing, wherein biological processes are slowed down thus allowing prolonged storage of biological material (e.g., below 8° C. and above 0° C., or between 4° C. and 8° C.).

The term “about” in reference to a numeric value refers to +10% of the stated numeric value. In other words, the numeric value can be in a range of 90% of the stated value to 110% of the stated value.

### Preservation

The present disclosure provides for preservation compositions and methods for preserving biological materials.

The present method comprises contacting/combining the viral vectors or viral particles with a preservation composition. In certain embodiment, this contacting/combining/mixing step involves adding the preservation composition to the viral vectors or viral particles, and mixing the viral vectors or viral particles with the preservation composition. This step of the present method (e.g., step (a)) may results in obtaining a mixture or combination (e.g., a liquid mixture or combination) of the viral vectors or viral particles in suspension in the composition.

The present disclosure provides a method of preserving viral vectors or viral particles comprising the steps of placing viral vectors or viral particles in the preservation composition, and preserving the viral vectors or viral particles that are in the preservation composition.

In certain embodiment, when viral vectors or viral particles are treated for cryopreservation, after the viral vectors or viral particles are suspended in the cryopreservation composition in a liquid state, the resulting suspension is frozen by maintaining it under conditions for cryopreservation. When the viral vectors or viral particles are needed, the frozen mixture of viral vectors or viral particles and the cryopreservation composition are subjected to a thawing process, after which the cells can be recovered.

The viral vectors or viral particles and the preservation composition can be physically combined according to a number of methods. In certain embodiments, the viral vectors or viral particles are present in a suspension prior to combination with the preservation composition. In one embodiment, the step of combining the viral vectors or viral particles with the preservation composition comprises providing the viral vectors or viral particles in a suspension and adding the preservation composition to the suspension (with or without mixing). In one embodiment, the step of combining the viral vectors or viral particles with the preservation composition comprises providing the viral vectors or viral particles in a suspension and adding the suspension to the cryopreservation composition (with or without mixing).

In certain embodiments, when or before being mixed/combined with the viral vectors or viral particles, the temperature of the preservation composition ranges from about 4° C. to about 45° C., from about 10° C. to about 40° C., from about 15° C. to about 40° C., from about 20° C. to about 40° C., from about 30° C. to about 40° C., from about 33° C. to about 38° C., or about 37° C.

In certain embodiments, the mixture of the viral vectors or viral particles and the cryopreservation medium is equilibrated prior to freezing the mixture. For example, the mixture is equilibrated for a time period ranging from about 10 seconds to about 1 hour, from about 20 seconds to about 50 minutes, from about 20 seconds to about 40 minutes, from about 30 seconds to about 30 minutes, from about 30 seconds to about 20 minutes, from about 30 seconds to about 10 minutes, from about 30 seconds to about 5 minutes, from about 30 seconds to about 2 minutes, from about 30 seconds to about 1 minute, from about 1 minute to about 40 minutes, from about 5 minutes to about 30 minutes, or from about 5 minutes to about 10 minutes.

In certain embodiments, prior to freezing the mixture, the mixture of the viral vectors or viral particles and the preservation composition is equilibrated at a temperature ranging from about 4° C. to about 45° C., from about 10° C. to about 40° C., from about 15° C. to about 40° C., from about 20° C. to about 40° C., from about 30° C. to about 40° C., from about 33° C. to about 38° C., or about 37° C.

In certain embodiments, the present method comprises the steps of combining viral vectors or viral particles with the present preservation composition, and subjecting the combined biological material and the present preservation composition to preservation conditions (e.g., cryopreservation conditions). In one embodiment, cryopreservation conditions can refer to an environment providing a cryopreservation temperature, or a temperature sufficiently below 0° C. In specific embodiments, a cryopreservation temperature comprises a temperature of at or below about 0° C., at or below about −20° C., at or below about −50° C., at or below about −60° C., at or below about −70° C., at or below about −80° C., at or below about −90° C., at or below about −100° C., at or below about −110° C., at or below about −120° C., at or below about −135° C., at or below about −196° C., or in liquid nitrogen.

Freeze-drying and cryopreservation in accordance with the present disclosure may be carried out in any method suitable to the biological material. The freezing of above methods of the present disclosure can be done in any method or apparatus known in the art.

In one embodiment, the viral vectors or viral particles are suspended in the preservation composition; the suspension thus prepared is dispensed into tubes or vials, and the resulting tubes or vials are placed in a storage container (e.g., a refrigerator, freezer, etc.) for storage.

In one embodiment, the viral vectors or viral particles are suspended in the cryopreservation composition; the suspension thus prepared is dispensed into freezing tubes (e.g., cryotubes, cryovials, etc.), and the resulting tubes are placed directly in an ultra-low temperature freezer (e.g., at −80° C.) to freeze the viral vectors or viral particles. In one embodiment, the viral vectors or viral particles in the cryopreservation composition is frozen directly in a freezer at −80° C.

The viral vectors or viral particles in the preservation composition may be stored for any length of time until they are needed. When the viral vectors or viral particles are at the storage temperature, they may be stored for a desired period, such as about 1-5 hours, about 5-12 hours, about 12-24 hours, about 24-48 hours, about 48 hours, about 1 week, about 2 weeks, about 3 weeks, about 1 month, about 2 months, about 3 months, about 6 months, about 1 year, about 2 years, about 3 years, about 4 years, about 5 years, or longer.

### Viral Vectors

The present preservation compositions may be used to stabilize/preserve adenoviruses, adeno-associated viruses, retroviruses, herpes viruses, etc.

In one embodiment, the viral vector is an adenoviral vector. In another embodiment, the viral vector is an adeno-associated viral vector. In yet another embodiment, the viral vector is a retroviral vector, such as a lentiviral vector.

Non-limiting examples of retroviruses include, lentivirus, Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV). Retroviruses may include, Alpharetrovirus (e.g., Avian leukosis virus); Betaretrovirus (e.g., Mouse mammary tumor virus); Gammaretrovirus (e.g., Murine leukemia virus); Deltaretrovirus (e.g., Bovine leukemia virus); Epsilonretrovirus (e.g., Walleye dermal sarcoma virus); Lentivirus (e.g., Human immunodeficiency virus 1); and Spumavirus (e.g., Chimpanzee foamy spumavirus). Retroviruses may include, Oncovirinae (e.g., Moloney murine leukemia virus (MoMOLV), Moloney murine sarcoma virus (MoMSV), and Mouse mammary tumor virus (MMTV), Spumavirinae, amd Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

Non-limiting examples of lentiviral vectors include human immunodeficiency virus (HIV); visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, the lentiviral vector is HIV-1. In another embodiment, the lentiviral vector is HIV-2. Any subtype, serotype and pseudotype of lentiviruses, and both naturally occurring and recombinant forms, may be used as a viral vector. Lentiviral vectors may include, without limitation, primate lentiviruses, goat lentiviruses, sheep lentiviruses, horse lentiviruses, cat lentiviruses, and cattle lentiviruses.

The viral vector may comprise an exogenous polynucleotide sequence.

In certain embodiments, the viral vector comprises a polynucleotide sequence encoding a chimeric antigen receptor (CAR) polypeptide, an engineered T cell receptor polypeptide, or a bi-specific T cell engager polypeptide.

The terms “retrovirus” and “retroviral vector” may be used interchangeably herein. The terms “lentivirus” and “lentiviral vectors” may be used interchangeably herein.

### Methods of Use

The present composition when comprising viral vectors may be used to transduce cells, to provide gene therapy, or to provide a vaccine.

In certain embodiments, recombinant viral vectors stabilized in the present preservation composition can be to infect/transduce cells in vitro or in vivo. In certain embodiments, the composition may be used for in vivo injection. In another embodiment, the composition may be used for in vitro use.

In certain embodiments, the composition is diluted at least 10× prior to in vivo injection or in vitro use. In certain embodiments, the composition is diluted at least 50× prior to in vivo injection or in vitro use. In another embodiment, the composition is diluted at least 100× prior to in vivo injection or in vitro use. In certain embodiments, the composition is diluted at least 200× prior to in vivo injection or in vitro use. In certain embodiments, the composition is diluted at least 250× prior to in vivo injection or in vitro use.

The present disclosure provides a method of treating a disease in a subject in need thereof. The method may comprise administering to the subject a population of cells transduced with the present composition (with viral vectors).

The present disclosure provides a method of transducing a cell with the present composition which also contains the viral vectors. The method may comprise contacting a population of cells with the present composition (with viral vectors).

The cell may be a mammalian cell, such as a human cell. The cell may be a stem cell, progenitor cell, or a differentiated cell. The cell may be a hematopoietic cell, including but not limited to, hematopoietic stem cells, CD34-expressing cells, hematopoietic progenitor cells, myeloid cells, lymphoid cells, B and T lymphocytes, and the like. In one embodiment, the hematopoietic cell is a hematopoietic stem or progenitor cell or a cell that expresses CD34. In another embodiment, the hematopoietic cell is a lymphocyte, such as a T lymphocyte (T cell), and a B cell. The cell may be an immune cell, such as a natural killer (NK) cell, a lymphocyte, a neutrophil, an eosinophil, a basophil, a mast cell, a monocyte, a macrophage, and a dendritic cell.

In certain embodiments, the cells are embryonic stem cells, induced pluripotent stem cells, bone marrow stem cells, umbilical cord stem cells, placental stem cells, mesenchymal stem cells, neural stem cells, liver stem cells, pancreatic stem cells, pancreatic endoderm, cardiac stem cells, kidney stem cells and hematopoietic stem cells.

The cell (e.g., immune cell) may be allogeneic or autologous. The mammalian recipient may be a human and the cell can be autologous with respect to the recipient. Alternatively, the cells can be allogeneic, syngeneic or xenogeneic with respect to the recipient.

The cells may be mammalian cells, e.g., from a rodent, a cat, a dog, a pig, a goat, a sheep, a horse, a cow, or a primate. In one embodiment, the cells are human cells. Suitable cells include fetal cells and adult cells.

The aqueous composition comprising viral vectors can be administered by direct injection to a cell, tissue, or organ of a subject in need of gene therapy, in vivo. In various other embodiments, cells are transduced in vitro or ex vivo with the present compositions when comprising viral vectors, and optionally expanded ex vivo. The transduced cells are then administered to a subject in need thereof.

In certain embodiments, the titers of the viral vectors may be at least or about 10⁵ transducing units per milliliter (TU/mL), at least or about 10⁶ TU/mL, at least or about 10⁷ TU/mL, at least or about 10⁸ TU/mL, at least or about 10⁹ TU/mL, at least or about 10¹⁰ TU/mL, at least or about 10¹¹ TU/mL, at least or about 10¹² TU/mL, or at least or about 103 TU/mL.

### Kits

The present disclosure also provides for a kit comprising the present preservation composition (in solid or liquid form as described herein). Such kits may include one or more containers comprising the present preservation composition, such as a first preservation reagent and a second preservation reagent. In one embodiment, the kit comprises the present preservation composition which comprises a first preservation reagent and a second preservation reagent.

In some embodiments, the kit can comprise instructions for use in any of the methods described herein. In one embodiment, the kit comprises instructions for preservation of viral vectors using the preservation composition and method. The kit may further comprise a description of selecting a subject suitable for treatment based on identifying whether the subject is in need of the treatment. In some embodiments, the instructions comprise a description of administering the thawed biological material after cryopreservation to a subject who is in need of the treatment. In certain embodiments, instructions supplied in the kits are written instructions on a label or package insert. The label or package insert may also indicate clinical and/or research applications of the biological material.

Parts of a kit may be used simultaneously or chronologically staggered, i.e., at different points in time and with equal or different time intervals for any component of a kit. Time intervals can be selected to obtain the desired effect.

The kits provided herein are in suitable packaging. Suitable packaging includes, but is not limited to, a vial (e.g., a cryovial), a bottle, an ampoule, a tube (e.g., a cryotube), a bag, a flask, a jar, flexible packaging, and the like. Also contemplated are packages for use in combination with a specific device, such as a storage tube, a storage vial, a freezing container, a cryovial and/or a cryotube.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

With extensive and in-depth research, the inventors of the present application have developed a low endotoxic viral vector preservation combination reagent. It is suitable for the production process of GMP-grade lentiviral vectors, can prolong the validity period of lentiviral preservation, and leads to a stable preparation process of high-purity and high-quality recombinant lentiviral vectors. It can be used to perform linear amplification, stably and continuously improve the production process, effectively control the cost, and perfect the production technology. In addition, it can help the preparation and clinical use of CAR-T cell products. On this basis, the present invention has been completed.

### Combination Reagent

The combination reagent of the present application may include component A and component B.

Component A may include: water, sucrose, potassium dihydrogen phosphate (KH₂PO₄), disodium hydrogen phosphate heptahydrate (Na₂HPO₄.7H₂O), and sodium chloride, in which a concentration of sucrose is 10-25 g/100 mL, a concentration of potassium dihydrogen phosphate (KH₂PO₄) is 120-150 mg/L, a concentration of disodium hydrogen phosphate heptahydrate (Na₂HPO₄.7H₂O) is 700-850 mg/L, and a concentration of sodium chloride is 8500-10000 mg/L.

Component A may have a pH of 6.95-7.45.

Component B may include: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which a concentration of sucrose is 10-25 g/100 mL, a concentration of human albumin is 4-30 g/100 mL, a concentration of sodium caprylate is 4-20 mmol/L, a concentration of sodium N-acetyltryptophan is 4-20 mmol/L, a concentration of potassium dihydrogen phosphate (KH₂PO₄) is 20-150 mg/L, a concentration of disodium hydrogen phosphate heptahydrate (Na₂HPO₄.7H₂O) is 150-800 mg/L, and a concentration of sodium chloride is 1500-10000 mg/L. Component B may have a pH of 6.95-7.45.

In certain embodiments, component A is used in the production and purification process of high-purity and high-quality lentiviral vectors; component B is added in a certain proportion before aliquoting so as to finally form the preservation solution of lentiviral vectors. In certain embodiments, a volume ratio of component A:component B, when in use, is within a range of from 4:1 to 1:1.

After the component B is added, the osmotic pressure of the obtained preservation solution may be 900-1400 mOsmol/Kg, and the pH thereof may be 6.95-7.45. In certain embodiments, the concentration of sucrose is 10-25 g/100 mL, the concentration of human albumin is 2-15 g/100 mL, the concentration of sodium caprylate is 1.6-10 mmol/L, the concentration of sodium N-acetyltryptophan is 1.6-10 mmol/L, the concentration of potassium dihydrogen phosphate (KH₂PO₄) is 70-130 mg/L, the concentration of disodium hydrogen phosphate heptahydrate (Na₂HPO₄.7H₂O) is 450-800 mg/L, and the concentration of sodium chloride is 5500-10000 mg/L.

The obtained liquid can preserve the lentiviral vector for a long period of time, and there is no obvious drop in the titer.

The combination reagent of the present invention has a better preservation effect on lentiviral vectors than commercially available products of the same type, and is suitable for long-term stable preservation of the second and third generations of lentiviral vectors.

The present invention will be further explained below in conjunction with specific embodiments. It should be understood that these embodiments are only used to illustrate the present invention, and not to limit the scope of the present invention. The experimental methods without specific conditions provided in the following examples are usually in accordance with the conventional conditions (such as the conditions described in Sambrook et al., Molecular Cloning: Laboratory Manual (New York: Cold Spring Harbor Laboratory Press, 1989)) or in accordance with the conditions recommended by the manufacturers. Unless otherwise stated, the percentages and parts herein are weight percentages and parts by weight.

The following are examples of the present invention and are not to be construed as limiting.

### Example 1

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 10 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mg/L. The pH is 7.15-7.25.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 10 g/100 mL, the concentration of human albumin is 12 g/100 mL, the concentration of sodium caprylate is 9.6 mmol/L, the concentration of sodium N-acetyltryptophan is 9.6 mmol/L, the concentration of potassium dihydrogen phosphate (KH₂PO₄) is 28.8 mg/L, the concentration of disodium hydrogen phosphate heptahydrate (Na₂HPO₄.7H₂O) is 167 mg/L, and the concentration of sodium chloride is 1800 mg/L. The pH is 7.15-7.25.

### Example 2

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 10 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mg/L. The pH is 7.35-7.45.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 10 g/100 mL, the concentration of human albumin is 24 g/100 mL, the concentration of sodium caprylate is 19.2 mmol/L, the concentration of sodium N-acetyltryptophan is 19.2 mmol/L, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mg/L. The pH is 7.35-7.45.

### Example 3

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mg/L. The pH is 7.15-7.25.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of human albumin is 6 g/100 mL, the concentration of sodium caprylate is 4.8 mmol/L, the concentration of sodium N-acetyltryptophan is 4.8 mmol/L, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 1800 mg/L. The pH is 7.15-7.25.

### Example 4

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9500 mg/L. The pH is 7.35-7.45.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of human albumin is 12 g/100 mL, the concentration of sodium caprylate is 9.58 mmol/L, the concentration of sodium N-acetyltryptophan is 9.58 mmol/L, the concentration of potassium dihydrogen phosphate (KH₂PO₄) is 28.8 mg/L, the concentration of disodium hydrogen phosphate heptahydrate (Na₂HPO₄.7H₂O) is 167.0 mg/L, and the concentration of sodium chloride is 1800 mg/L. The pH is 7.35-7.45.

### Example 5

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9500 mg/L. The pH is 7.05-7.15.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 15 g/100 mL, the concentration of human albumin is 24 g/100 mL, the concentration of sodium caprylate is 19.2 mmol/L, the concentration of sodium N-acetyltryptophan is 19.2 mmol/L, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mmol/L. The pH is 7.05-7.15.

### Example 6

A combination reagent includes a component A and a component B.

The component A includes: water, sucrose, potassium dihydrogen phosphate (KH₂PO₄), disodium hydrogen phosphate heptahydrate (Na₂HPO₄.7H₂O), and sodium chloride, in which the concentration of sucrose is 20 g/100 mL, the concentration of potassium dihydrogen phosphate (KH₂PO₄) is 144 mg/L, the concentration of disodium hydrogen phosphate heptahydrate (Na₂HPO₄.7H₂O) is 795 mg/L, and the concentration of sodium chloride is 9000 mg/L. The pH is 7.35-7.45.

The component B includes: water, sucrose, human albumin, sodium caprylate, sodium N-acetyl tryptophan, potassium dihydrogen phosphate (KH₂PO₄), disodium hydrogen phosphate heptahydrate (Na₂HPO₄.7H₂O), and sodium chloride, in which the concentration of sucrose is 20 g/100 mL, the concentration of human albumin is 6 g/100 mL, the concentration of sodium caprylate is 4.8 mmol/L, the concentration of sodium N-acetyltryptophan is 4.8 mmol/L, the concentration of potassium dihydrogen phosphate (KH₂PO₄) is 28.8 mg/L, the concentration of disodium hydrogen phosphate heptahydrate (Na₂HPO₄.7H₂O) is 167.0 mg/L, and the concentration of sodium chloride is 9000 mg/L. The pH is 7.35-7.45.

The component A and component B prepared according to the compositions described in Embodiments 1-6 are used separately. The component A is used in the production and purification process of high-purity and high-quality lentiviral vectors, while the component B is added before aliquoting so as to finally form a preservation solution for lentiviral vectors. The volume ratio of component A:component B is between 4:1 and 1:1. The osmotic pressure of the preservation solution obtained after mixing is 900-1400 mOsmol/Kg, and the pH thereof is 6.95-7.45. The concentration of sucrose is 10-25 g/100 mL, the concentration of human albumin is 2-15 g/100 mL, the concentration of sodium caprylate is 1.6-10 mmol/L, the concentration of sodium N-acetyltryptophan is 1.6-10 mmol/L, the concentration of potassium dihydrogen phosphate (KH₂PO₄) is 70-130 mg/L, the concentration of disodium hydrogen phosphate heptahydrate (Na₂HPO₄.7H₂O) is 450-800 mg/L, and the concentration of sodium chloride is 5500-10000 mg/L.

### Example 7

Accelerated Degradation Test

After the same lentivirus CBMG-LRA2 is subjected to accelerated inactivation at 37° C. in different storage solutions prepared in Embodiments 1-6, the rate of decrease of lentivirus biotiter is detected.

293T cells are resuscitated and then plated on a culture dish, the cells are cultured for two days and then passaged, the passaged cells are cultured for 20-24 hours and then are transfected by means of multi-plasmid co-transfection 6 hours after the transfection, the culture medium is replaced, and the cells are further cultured for 72 hours. Next, the supernatant with the lentiviral vector is collected, and the collected supernatant is centrifuged and filtered with a filter of 0.45 μm to remove cell debris. The filtered lentivirus is aliquoted to ultracentrifuge tubes and then ultracentrifuged at 20000 g at 4 degrees for 90 minutes. The lentivirus pellets are respectively re-suspended in 1 ml of the preservation solution as show in Table 1, so as to dissolve the pellets at 4° C. overnight. The next day, the resulting solution is aliquoted to tubes with 150 μl per tube. The tubes are quickly put in dry ice to freeze and then stored in a refrigerator at −80 degrees. The lentiviruses in different preservation solutions after being frozen for 24 hours are then thawed in a 37° C. water bath. The operation is performed according to QC-SOP-090 lentiviral vector biological titer determination (ProteinL method) standard operating procedures SOP, and 0 hr titer detection is then performed. The virus dissolved in water bath is continuously maintained at a temperature of 37 degrees for 44-48 hours, and then detected again.

In the test, the combination reagent of the present invention is compared with products of the same type on the market (Table 1); it can be seen that the performance result of the combination reagent of the present invention is much better than those of the same type products on the market.

TABLE 1
Lentivirus titer preservation rate

| | Titer (TU/mL) | | Pre- |
|---|---|---|---|
| | 0 hr (start | | servation |
| Reagent source | time point) | 44-48 hr | rate (%) |
| Combination reagent in Example 1 | 1.17E+07 | 2.75E+06 | 23.59 |
| Combination reagent in Example 2 | 1.26E+07 | 1.80E+06 | 14.27 |
| Combination reagent in Example 3 | 1.24E+07 | 3.12E+06 | 25.11 |
| Combination reagent in Example 4 | 2.87E+07 | 1.64E+06 | 5.69 |
| Combination reagent in Example 5 | 2.91E+07 | 1.07E+07 | 36.72 |
| Combination reagent in Example 6 | 9.28E+06 | 1.38E+06 | 14.88 |
| Umibio (Shanghai) Co., Ltd. | 3.34E+07 | 1.14E+06 | 3.41 |
| Shenzhen Yuanxing Gene | 1.36E+07 | 2.71E+06 | 19.92 |
| Technology Co., Ltd. | | | |

Long-Term Stability Test

The combination reagent is used in the experiments for tracking the long-term storage effect of GMP-grade lentiviral vectors. The lentiviral vectors are frozen and stored at −80° C., and after being stored for a certain period of time, they are then thawed and the biological titer thereof is then detected. For the lentiviral vectors prepared by the second-generation or third-generation systems, there is currently no significant drop in titer (Table 2).

TABLE 2
Lentiviral vector stability test results

| Time | Titer (TU/mL) | | |
|---|---|---|---|
| (Test point/ | Second-generation | Third-generation lentiviral | |
| month) | lentiviral vector | vector | |
| Start (0) | 1.25 × 10⁸ | l.01 × 10⁸ | 1.43 × 10⁸ |
| 3 | 1.42 × 10⁸ | — | — |
| 6 | — | 1.63 × 10⁸ | 2.29 × 10⁸ |

The above experimental results show that the combination reagent of the present invention has better effects on lentiviral vectors than commercially available products of the same type, and is suitable for long-term stable storage of second- and third-generation lentiviral vectors.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A preservation composition for preserving viral vectors, comprising a first preservation reagent and a second preservation reagent,
wherein the first preservation reagent comprises sucrose at a concentration of 100 g/L to 250 g/L, potassium dihydrogen phosphate or sodium dihydrogen phosphate at a concentration of 0.8 mmol/L to 1.2 mmol/L, disodium hydrogen phosphate or dipotassium hydrogen phosphate at a concentration of 2.6 mmol/L-3.2 mmol/L, and sodium chloride at a concentration of 8.5-10 g/L,
wherein the second preservation reagent comprises sucrose at a concentration of 100 g/L to 250 g/L, albumin at a concentration of 40 g/L to 300 g/L, sodium caprylate or potassium caprylate at a concentration of 4 mmol/L to 20 mmol/L, sodium N-acetyltryptophan or potassium N-acetyltryptophan at a concentration of 4 mmol/L to 20 mmol/L, potassium dihydrogen phosphate or sodium dihydrogen phosphate at a concentration of 0.1 mmol/L-1.2 mmol/L, disodium hydrogen phosphate or dipotassium hydrogen phosphate at a concentration of 0.5 mmol/L-3 mmol/L, and sodium chloride at a concentration of 1.5-10 g/L.

2. The preservation composition of claim 1, comprising sucrose at a concentration of 100 g/L to 250 g/L, albumin at a concentration of 20 g/L to 150 g/L, sodium caprylate or potassium caprylate at a concentration of 1.6 mmol/L to 10 mmol/L, sodium N-acetyltryptophan or potassium N-acetyltryptophan at a concentration of 1.6 mmol/L to 10 mmol/L, potassium dihydrogen phosphate or sodium dihydrogen phosphate at a concentration of 0.5 mmol/L to 1 mmol/L, disodium hydrogen phosphate or dipotassium hydrogen phosphate at a concentration of 1.6 mmol/L to 3 mmol/L, and sodium chloride at a concentration of 5.5-10 g/L.

3. The preservation composition of claim 1, wherein the first preservation reagent and the second preservation reagent have a volume ratio ranging from about 1:1 to about 4:1.

4. The preservation composition of claim 1, wherein the first preservation reagent and the second preservation reagent are separate or mixed.

5. The preservation composition of claim 1, having an osmotic pressure ranging from about 900 mOsmol/kg to about 1400 mOsmol/kg.

6. The preservation composition of claim 1, having a pH ranging from about 6.95 to about 7.45.

7. The preservation composition of claim 1, further comprising viral vectors.

8. The preservation composition of claim 1, wherein the viral vectors are retroviral vectors.

9. The preservation composition of claim 1, wherein the retroviral vectors are lentiviral vectors.

10. The preservation composition of claim 1, wherein the lentiviral vectors are selected from the group consisting of human immunodeficiency virus (HIV); visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV).

11. The preservation composition of claim 1, wherein the lentiviral vectors are HIV-1 or HIV-2.

12. A method for preserving viral vectors, the method comprising combining the preservation composition of claim 1 with the viral vectors.

13. A method for preserving viral vectors using the preservation composition of claim 1, the method comprising:
(a) combining the first preservation reagent with the viral vectors to form a mixture; and
(b) combining the second preservation reagent with the mixture of step (a).

14. The method of claim 13, wherein the first preservation reagent and the second preservation reagent have a volume ratio ranging from about 1:1 to about 4:1.

15. A method of transducing a cell, the method comprising contacting the cell with the preservation composition of claim 7.

16. The method of claim 15, wherein the cell is a human cell.

17. The method of claim 15, wherein the cell is a stem cell or progenitor cell.

18. The method of claim 15, wherein the cell is a hematopoietic cell.

19. The method of claim 18, wherein the hematopoietic cell is a lymphocyte.

20. The method of claim 19, wherein the lymphocyte is a T lymphocyte.
